(19) United States
(12) Patent Application Publication
(10) Pub. No.: US 2026/0364028 A1
(43) Pub. Date: Nov. 19, 2026

(54) COMBINATION TREATMENTS FOR DEPRESSION AND OTHER DISORDERS

(71) Applicant: NEURAWELL THERAPEUTICS, Sunny isles Beach, FL (US)

(72) Inventors: Magid Abraham, Sunny Isles Beach, FL (US); Kelsey Satterly, Seattle, WA (US); Jay Mason, Reno, NV (US)

(21) Appl. No.: 19/488,341

(22) PCT Filed: Jun. 17, 2024
(86) PCT No.: PCT/US2024/034303

§ 371 Date: Nov. 26, 2025

Related U.S. Application Data
(60) Provisional application No. 63/521,578, filed on Jun. 16, 2023.
(60) Provisional application No. 63/528,285, filed on Jul. 21, 2023.
(60) Provisional application No. 63/638,117, filed on Apr. 24, 2024.

(51) Int. Cl. A61K 31/135; A61K 31/138; A61K 31/165; A61K 31/403; A61K 45/06; A61P 25/24

(52) U.S. Cl. CPC A61K 31/135; A61K 31/138; A61K 31/165; A61K 31/403; A61K 45/06; A61P 25/24

(57) ABSTRACT

The invention provides dosage forms that contain both a monoamine oxidase inhibitor (MAOI) and a beta blocker (e.g., propranolol, metoprolol, carvedilol, timolol, carteolol, atenolol, nebivolol, sotalol, bucindolol, nadolol, celiprolol, nebivolol, betaxolol, esmolol, bisoprolol, oxprenolol, penbutolol, labetalol, acebutolol, and pindolol) and methods of making and using same in, for example, for the treatment of psychiatric disorders (e.g., depression, major depressive disorder, major depressive disorder with anxious distress, treatment resistant depression, anxious depression, and mixed anxiety and depression). This abstract is intended as a scanning tool for purposes of searching in the particular art and is not intended to be limiting of the present invention.

### CROSS-REFERENCE TO RELATED APPLICATIONS

This application claims the benefit of U.S. Application No. 63/521,578, filed on Jun. 16, 2023, U.S. Application No. 63/528,285, filed on Jul. 21, 2023, and U.S. Application No. 63/638,117, filed on Apr. 24, 2024, the contents of which are incorporated herein by reference in their entireties.

### BACKGROUND

Depression is a leading cause of disability worldwide (Friedrich, M. J. (2017) JAMA 317(15): 1517), yet few novel antidepressants have been approved, and shortcomings in the effectiveness of antidepressant treatments contribute to the enormous public health burden of depression (Hyman, Steven E. (2012) Science translational medicine 4(155): 155 cm11). Major depressive disorder (MDD) is the most prevalent and disabling form of depression, and the lifetime prevalence of MDD in the United States (US) is 20.6% (Hasin et al. (2018) JAMA Psychiatry 75(4): 336-346). MDD frequently appears with other comorbidities in patients, including the presence of anxiety disorders. Indeed, the prevalence of anxiety or anxious symptoms in MDD is estimated to be between 40-78% (Thase et al. (2017) J Clin Psychiatry 78(9): 1351-1362; Gaspersz et al. (2017) J Clin Psychiatry 78(2): 207-213; Zimmerman et al. (2019) Depress Anxiety 36(1): 31-38; Fava et al. (2006) Journal of Psychiatric Research 40(4): 328-336; Yang et al. (2014) Psychiatry Clin Neurosci 68(9): 712-720. For some patients, the coexistence of depression and anxiety symptoms reflects an ongoing anxiety disorder, while for others, a significant surge in anxiety occurs with depressive episodes and complicates the clinical picture and treatment (Thase et al. (2017) J Clin Psychiatry 78(9): 1351-1362). Such patients, having both MDD and anxious distress (MDD-AD), tend to have more severe depression, severe functional impairment, increased panic and stress, and decreased response to treatment (Trivedi et al. (2006) The American journal of psychiatry 163(1): 28-40; Rosellini et al. (2018) Journal of Psychiatric Research 103:54-60; Hasin et al. (2018) JAMA Psychiatry 75(4): 336-346).

There is no standardized care for patients with MDD-AD. Research and clinical practice have shown monoamine oxidase inhibitors (MAOIs) to be effective in treating MDD when other treatments have failed (for review, see Bender and Walker, 2012; Fiedorowicz and Swartz, 2004; and Thase, et al., 1995). As demonstrated by McGrath et al. 1993, one MAOI, phenelzine (PHZ), is shown to be exceptionally effective at treating refractory depression, with 55% of patients previously unresponsive to the tricyclic antidepressant (TCA), imipramine, traditionally used as a benchmark for antidepressant effectiveness, responding positively to PHZ (McGrath et al. (1993) The American journal of psychiatry 150(1): 118-123). Furthermore, a recent meta-analysis comparing 14 antidepressants (and placebo) demonstrated that PHZ has superior efficacy compared to all other treatments (Suchting et al. (2021) Journal of affective disorders 282:1153-1160). Phenelzine sulfate is proposed to increase gamma-aminobutyric acid (GABA) brain levels through inhibition of GABA transaminase (GABA-T), an inhibitory neurotransmitter widely accepted to play a role in anxiety disorders (Nemeroff, Charles B. (2003) Psychopharmacol Bull 37(4): 133-146). The potentiation of GABA, serotonin, norepinephrine, and dopamine by PHZ, all of which are proposed to play a role in mediating depression and anxiety disorders, may explain the reported efficacy of PHZ and its potential for treating MDD-AD.

Despite success with treating difficult-to-treat patients, PHZ and other MAOIs are rarely used to treat MDD, mixed anxiety and depression states, or MDD-AD, even after several treatment options have failed, primarily due to safety concerns related to accumulation of tyramine (the “cheese effect”). In patients administering MAOIs during the 1960s, tyramine was shown to be responsible for many medically significant cardiovascular events (e.g., dangerously high blood pressure), and 21 fatalities (Gillman, P. Ken (2011) Journal of psychopharmacology (Oxford, England) 25(3): 429-436). After a temporary withdrawal from the market in 1964, MAOIs were reinstated with utility-limiting dietary restrictions imposed by regulators to prevent the “cheese effect,” including limitations on consumption of cheese, beer, wine, and preserved meat. After 1967, it was reported to be difficult to identify reports of fatalities related to the cheese effect (Gillman, P. Ken (2011) Journal of psychopharmacology (Oxford, England) 25(3): 429-436). The potential for tyramine effects, however, continues to be reported as a significant deterrent to the use of MAOIs today (Rabkin et al. (1985) J. Clin. Psychopharmacol. 5(1): 2-9) with concern by prescribers of the dangers of patient lapses in compliance, misunderstanding of the risks of dietary non-compliance, and the potential medical risks consequent to a dangerous hypertensive reaction among patients who deviate from the diet restrictions.

Accordingly, there remains a need for dosage forms, compositions, and methods of treating depression and other psychiatric disorders using MAOIs that reduce or even eliminate the tyramine-induced pressor response. These needs and others are met by the present invention.

### SUMMARY

In accordance with the purpose(s) of the invention, as embodied and broadly described herein, the invention, in one aspect, relates to dosage forms that contain both a MAOI and a beta blocker (e.g., propranolol, metoprolol, carvedilol, timolol, carteolol, atenolol, nebivolol, sotalol, bucindolol, nadolol, celiprolol, nebivolol, betaxolol, esmolol, bisoprolol, oxprenolol, penbutolol, labetalol, acebutolol, and pindolol) and methods of making and using same in the treatment of psychiatric disorders such as, for example, depression, major depressive disorder, major depressive disorder with anxious distress, treatment resistant depression, anxious depression, and mixed anxiety and depression.

Thus, disclosed are dosage forms comprising: (a) a therapeutically effective amount of a monoamine oxidase inhibitor (MAOI); and (b) a therapeutically effective amount of a beta blocker selected from propranolol, metoprolol, carvedilol, timolol, carteolol, atenolol, nebivolol, sotalol, bucindolol, nadolol, celiprolol, nebivolol, betaxolol, esmolol, bisoprolol, oxprenolol, penbutolol, labetalol, acebutolol, and pindolol or a pharmaceutically acceptable salt or free base thereof, and a pharmaceutically acceptable carrier.

Also disclosed are dosage forms comprising: (a) a therapeutically effective amount of phenelzine or a pharmaceutically acceptable salt or free base thereof; and (b) a therapeutically effective amount of a beta blocker selected from pindolol and bisoprolol, or a pharmaceutically acceptable salt or free base thereof, and a pharmaceutically acceptable carrier, wherein the MAOI is formulated as a modified-release dosage form, and wherein the beta blocker is formulated as a modified-release dosage form.

Also disclosed are dosage forms comprising a therapeutically effective amount of a MAOI, the improvement comprising further including therapeutically effective amount of a beta blocker selected from propranolol, metoprolol, carvedilol, timolol, carteolol, atenolol, nebivolol, sotalol, bucindolol, nadolol, celiprolol, nebivolol, betaxolol, esmolol, bisoprolol, oxprenolol, penbutolol, labetalol, acebutolol, and pindolol or a pharmaceutically acceptable salt or free base thereof, in the dosage form.

Also disclosed are dosage forms comprising a therapeutically effective amount of a beta blocker, the improvement comprising further including a therapeutically effective amount of MAOI in the dosage form.

Also disclosed are methods for treating a psychiatric disorder in a subject in need thereof, the method comprising administering to the subject an effective amount of a disclosed dosage form.

Also disclosed are methods for treating a psychiatric disorder in a subject in need thereof, the method comprising administering to the subject: (a) a therapeutically effective amount of a monoamine oxidase inhibitor (MAOI); and (b) a therapeutically effective amount of a beta blocker selected from propranolol, metoprolol, carvedilol, sotalol, bucindolol, nadolol, celiprolol, nebivolol, betaxolol, esmolol, bisoprolol, oxprenolol, penbutolol, labetalol, acebutolol, and pindolol, or a pharmaceutically acceptable salt or free base thereof, wherein the subject has not previously been diagnosed as having migraines, and wherein the subject is not currently experiencing a migraine.

Also disclosed are methods for treating a psychiatric disorder in a subject in need thereof by administering a MAOI, the improvement comprising simultaneously administering a beta blocker selected from propranolol, metoprolol, carvedilol, timolol, carteolol, atenolol, nebivolol, sotalol, bucindolol, nadolol, celiprolol, nebivolol, betaxolol, esmolol, bisoprolol, oxprenolol, penbutolol, labetalol, acebutolol, and pindolol, or a pharmaceutically acceptable salt or free base thereof, to the patient in an amount that, together with the MAOI, is effective to reduce migraine relapse and/or produce longer lasting efficacy compared to the administration of the MAOI in the absence of the beta blocker, wherein the MAOI and the beta blocker are together present in a single dosage form.

Also disclosed are methods for treating a psychiatric disorder in a subject in need thereof by administering a MAOI, the improvement comprising simultaneously administering a beta blocker selected from propranolol, metoprolol, carvedilol, timolol, carteolol, atenolol, nebivolol, sotalol, bucindolol, nadolol, celiprolol, nebivolol, betaxolol, esmolol, bisoprolol, oxprenolol, penbutolol, labetalol, acebutolol, and pindolol, or a pharmaceutically acceptable salt or free base thereof, to the patient in an amount that, together with the MAOI, is effective to reduce migraine relapse and/or produce longer lasting efficacy compared to the administration of the MAOI in the absence of the beta blocker, wherein the subject has not previously been diagnosed as having migraines and wherein the subject is not currently experiencing a migraine.

Also disclosed are methods for treating a psychiatric disorder in a subject in need thereof by administering a beta blocker, the improvement comprising simultaneously administering a MAOI to the patient in an amount that, together with the beta blocker, is effective to reduce migraine relapse and/or produce longer lasting efficacy compared to the administration of the beta blocker in the absence of the MAOI, wherein the MAOI and the beta blocker are together present in a single dosage form.

Also disclosed are methods for treating a psychiatric disorder in a subject in need thereof by administering a beta blocker, the improvement comprising simultaneously administering a beta blocker selected from propranolol, metoprolol, carvedilol, timolol, carteolol, atenolol, nebivolol, sotalol, bucindolol, nadolol, celiprolol, nebivolol, betaxolol, esmolol, bisoprolol, oxprenolol, penbutolol, labetalol, acebutolol, and pindolol, or a pharmaceutically acceptable salt or free base thereof, to the patient in an amount that, together with the MAOI, is effective to reduce migraine relapse and/or produce longer lasting efficacy compared to the administration of the MAOI in the absence of the beta blocker, wherein the subject has not previously been diagnosed as having migraines and wherein the subject is not currently experiencing a migraine.

Also disclosed are methods for treating a psychiatric disorder in a subject in need thereof, the method comprising administering to the subject an effective amount of a dosage form comprising: (a) a therapeutically effective amount of a monoamine oxidase inhibitor (MAOI) selected from phenelzine and tranylcypromine (TCP); (b) a therapeutically effective amount of a beta blocker selected from pindolol and carvedilol, or a pharmaceutically acceptable salt or free base thereof; and (c) a pharmaceutically acceptable carrier, wherein the subject has not previously been diagnosed as having migraines.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE DRAWINGS

The accompanying figures, which are incorporated in and constitute a part of this specification, illustrate several aspects and together with the description serve to explain the principles of the invention.

FIG. 1 shows a representative schematic of a prophetic phase 1 study described herein.

FIG. 2 shows a representative data illustrating the IC₅₀ of MAO-A with phenelzine.

FIG. 3 shows a representative data illustrating the IC₅₀ of MAO-B with phenelzine.

FIG. 4A and FIG. 4B show representative data illustrating the maximum change in systolic blood of either phenylzine (FIG. 4A) or tranylcypromine (FIG. 4B) alone and in combination with pindolol on changes to blood pressure after a tyramine dose.

FIG. 5 shows a representative time course of systolic blood pressure in the rat for phenelzine alone or in combination with pindolol following administration of tyramine.

FIG. 6 shows a representative time course with tranylcypromine with pindolol following administration of tyramine.

FIG. 7 shows representative data illustrating the maximum change from baseline for phenelzine alone or in combination with pindolol.

FIG. 8 shows representative data illustrating the maximum change from baseline for tranylcypromine alone or in combination with either pindolol or bisoprolol.

FIG. 9 shows representative data illustrating the area under the curve (AUC) for tranylcypromine alone or in combination with either pindolol or bisoprolol.

FIG. 10 shows representative data illustrating the time course change from baseline for tranylcypromine alone or in combination with pindolol.

FIG. 11 shows representative data illustrating the time course change from baseline for tranylcypromine alone or in combination with bisoprolol.

FIG. 12 shows representative data illustrating the maximum change from baseline for tranylcypromine alone or in combination with carvedilol.

FIG. 13 shows representative data illustrating the AUC for tranylcypromine alone or in combination with carvedilol.

FIG. 14 shows representative data illustrating the time course change from baseline for tranylcypromine alone or in combination with carvedilol.

FIG. 15 shows representative data illustrating a comparison of the transient change from baseline of systolic blood pressure after dose administration of tranylcypromine compared to tranylcypromine with pindolol or bisoprolol.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein may be different from the actual publication dates, which can require independent confirmation.

### A. DEFINITIONS

As used in the specification and the appended claims, the singular forms “a,” “an” and “the” include plural referents unless the context clearly dictates otherwise. Thus, for example, reference to “an antidepressant,” “a psychological disorder,” or “a subject” includes mixtures of two or more such antidepressants, psychological disorders, or subjects, and the like.

As used in the specification and in the claims, the term “comprising” can include the aspects “consisting of” and “consisting essentially of.”

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” means that the subsequently described event or circumstance can or cannot occur, and that the description includes instances where said event or circumstance occurs and instances where it does not.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig, or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, the term “dosage form” means one or more pharmacologically active agents in a medium, carrier, excipient, binder, vehicle, or filler that is suitable for administration to a subject. A dosage form, as used herein, can refer to a liquid or a solid dosage form. A dosage form can comprise one or more disclosed compounds, a combination of the disclosed compounds, fixed dose drug combinations (FDCs), a product of a disclosed method of making, or a salt, solvate, free base, or polymorph thereof, in combination with a pharmaceutically acceptable excipient, such as a preservative, buffer, saline, or phosphate buffered saline or blended with pharmaceutically acceptable binding agents such as pregelatinized maze starch or hydroxypropyl methylcellulose of varying degrees of substitutions; fillers (e.g., lactose, microcrystalline cellulose, calcium carbonate or calcium phosphate); disintegrants (potato starch, croscarmellose Sodium, or Sodium starch glycolate); wetting agents (e.g., sodium lauryl sulphate or non-ionic surfactants) or other agents suitable for tableting or inactive ingredients for tablet, pellet, or spheres for matrix release including nonionic homopolymers of ethylene oxide, water soluble natural gums of polysaccharides water swellable but insoluble, high molecular weight homopolymers and copolymers of acrylic acid chemically crosslinked with polyalkenyl alcohols, polyvinyl acetate and povidones, crosslinked amylose starch and ionic methacrylate copolymers. Additionally fatty acids, fatty acid esters and mono-, di-, tri-glycerides of differing melting points as well as hydrophobic polymers, natural occurring waxes, and ammoniomethacrylate copolymers are used in non-swellable matrix. Lipid or hydrophobic matrices can delay the release of the drug and also are used in the delayed release technology. Coating materials for modified release of drug from formulated matrices (tablets, pellets, or spheres), which includes enteric and other release targets include shellac and zein of natural sources, derivatives of cellulose (cellulose acetate phthalate, hydroxypropyl methylcellulose phthalate) and methacrylic (acid and ester copolymers containing carboxylic functional groups) that resist solubility low pH and protect the delivery to other pH greater than 5.5. Plasticizers (such as triacetin, triethanol citrate, glycerin, and polyethylene glycol) may or may not be needed to facilitate the release profile and flexibility of the coating.

Dosage forms can be made using conventional pharmaceutical manufacturing and compounding techniques. Dosage forms can comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene9-10 nonyl phenol, sodium desoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsiloxane), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol). A dosage form formulated for injectable use can have a disclosed compound, a product of a disclosed method of making, or a salt, solvate, free base, or polymorph thereof, suspended in sterile saline solution for injection together with a preservative.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

As used herein, the terms “therapeutic agent” include any synthetic or naturally occurring biologically active compound or composition of matter which, when administered to an organism (human or nonhuman animal), induces a desired pharmacologic, immunogenic, and/or physiologic effect by local and/or systemic action. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14^{th} edition), the Physicians' Desk Reference (64^{th} edition), and The Pharmacological Basis of Therapeutics (12^{th} edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrhythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term “therapeutic agent” also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

The term “pharmaceutically acceptable” describes a material that is not biologically or otherwise undesirable, i.e., without causing an unacceptable level of undesirable biological effects or interacting in a deleterious manner.

As used herein, the term “pharmaceutically acceptable carrier” refers to non-sterile and sterile carrier or excipient that is generally safe (non-toxic) and is not considered biologically active itself, and may be part of an aqueous or nonaqueous solutions, dispersions, suspensions or emulsions, as well as sterile powders for reconstitution into sterile injectable solutions or dispersions just prior to use or non-sterile powders for oral administration. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions, which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the term “monoamine oxidase inhibitor(s)” or “MAOI(s)” means a compound that inhibits the activity of one or more monoamine oxidase enzymes (e.g., MAO-A, MAO-B). A MAOI can inhibit a MAO in a nonselective (e.g., inhibiting both MAO-A and MAO-B) or selective manner (e.g., inhibiting either MAO-A or MAO-B), and can also act in a reversible (inactivating MAO-A and/or MAO-B through a noncovalent, reversible interaction; e.g., competitive inhibitors, noncompetitive inhibitors) or in an irreversible (inactivating MAO-A and/or MAO-B through a covalent, irreversible interaction) manner. Exemplary MAOIs include, but are not limited by, tranylcypromine (TCP) (e.g., tranylcypromine sulfate, tranylcypromine hydrochloride), phenelzine (e.g., phenelzine sulfate), isocarboxazid, selegiline (e.g., selegiline hydrochloride), rasagiline (e.g., rasagiline mesylate), and moclobemide (e.g., moclobemide hydrochloride). Free base forms, pharmaceutically acceptable salt forms, and non-salt forms are contemplated.

The term “beta blocker(s)” means a compound that blocks beta-adrenergic signaling and is used to treat cardiovascular conditions including hypertension. As used herein, “beta blockers” include non-selective beta-blockers (e.g., propranolol, nadolol, pindolol, labetalol, penbutolol, sotalol, carvedilol, timolol), beta-1 selective blockers (e.g., metoprolol, atenolol, acebutolol, betaxolol, esmolol, bisoprolol, nebivolol), beta-blockers with intrinsic sympathomimetic activity (ISA) or partial agonism (e.g., acebutolol, pindolol, carteolol, penbutolol), and/or dual alpha and beta blockers (e.g., carvedilol, labetalol, dilevalol). Exemplary beta blockers include, but are not limited to, propranolol, metoprolol, carvedilol, timolol, carteolol, atenolol, nebivolol, sotalol, bucindolol, nadolol, celiprolol, nebivolol, betaxolol, esmolol, bisoprolol, oxprenolol, penbutolol, labetalol, acebutolol, and pindolol. Free base forms, pharmaceutically acceptable salt forms, and non-salt forms are contemplated.

The phrase “beta blocker with intrinsic sympathomimetic activity (ISA),” as used herein, refers to a beta blocker that can stimulate beta-adrenergic receptors (agonist effect) and also oppose the stimulating effects of catecholamines (antagonist effect) in a competitive way. Exemplary beta blockers with ISA include, but are not limited to, pindolol, carteolol, penbutolol, and acebutolol.

As used herein, the term “modified-release” refers to a dosage form in which the timing and/or rate of release of a drug substance(s) is altered as compared to that of a conventional, immediate release dosage form. A modified-release dosage form can include extended-release dosage forms, prolonged-release dosage forms, sustained-release dosage forms, controlled-release dosage forms, and delayed-release dosage forms. As described herein, a modified-release dosage form can be designed to achieve a prolonged therapeutic effect over an extended period of time after administration of a single dose. Thus, for example, in various aspects, a modified-release dosage form can release a drug substance (e.g., a MAOI) for a dissolution period lasting from about 6 hours to about 24 hours, from about 6 hours to about 20 hours, from about 6 hours to about 16 hours, from about 6 hours to about 12 hours, from about 6 hours to about 8 hours, from about 7 hours to about 24 hours, from about 8 hours to about 24 hours, from about 10 hours to about 24 hours, from about 12 hours to about 24 hours, from about 14 hours to about 24 hours, from about 16 hours to about 24 hours, from about 18 hours to about 24 hours, from about 20 hours to about 24 hours, from about 22 hours to about 24 hours, from about 8 hours to about 22 hours, from about 10 hours to about 20 hours, from about 12 hours to about 18 hours, or from about 14 hours to about 16 hours subsequent to administration of the dosage form to a patient. In various aspects, a modified-release dosage form can release a drug substance (e.g., a beta blocker) for a dissolution period lasting from about 6 hours to about 16 hours, from about 6 hours to about 14 hours, from about 6 hours to about 12 hours, from about 6 hours to about 10 hours, from about 6 hours to about 8 hours, from about 8 hours to about 16 hours, from about 10 hours to about 16 hours, from about 12 hours to about 16 hours, from about 14 hours to about 16 hours, from about 8 hours to about 14 hours, or from about 10 hours to about 12 hours subsequent to administration of the dosage form to a patient. In some aspects, a modified-release dosage form can contain more than one drug substance such that each drug substance is released over different dissolution periods. In some aspects, a modified-release dosage form can contain more than one drug substance such that each drug substance is released over approximately the same dissolution period.

In various further aspects, a modified-release dosage form can release a drug substance (e.g., a MAOI) for a absorption period lasting from about 6 hours to about 24 hours, from about 6 hours to about 20 hours, from about 6 hours to about 16 hours, from about 6 hours to about 12 hours, from about 6 hours to about 8 hours, from about 7 hours to about 24 hours, from about 8 hours to about 24 hours, from about 10 hours to about 24 hours, from about 12 hours to about 24 hours, from about 14 hours to about 24 hours, from about 16 hours to about 24 hours, from about 18 hours to about 24 hours, from about 20 hours to about 24 hours, from about 22 hours to about 24 hours, from about 8 hours to about 22 hours, from about 10 hours to about 20 hours, from about 12 hours to about 18 hours, or from about 14 hours to about 16 hours subsequent to administration of the dosage form to a patient. In various aspects, a modified-release dosage form can release a drug substance (e.g., a beta blocker) for a absorption period lasting from about 6 hours to about 16 hours, from about 6 hours to about 14 hours, from about 6 hours to about 12 hours, from about 6 hours to about 10 hours, from about 6 hours to about 8 hours, from about 8 hours to about 16 hours, from about 10 hours to about 16 hours, from about 12 hours to about 16 hours, from about 14 hours to about 16 hours, from about 8 hours to about 14 hours, or from about 10 hours to about 12 hours subsequent to administration of the dosage form to a patient. In some aspects, a modified-release dosage form can contain more than one drug substance such that each drug substance is released over different absorption periods. In some aspects, a modified-release dosage form can contain more than one drug substance such that each drug substance is released over approximately the same absorption period.

As used herein, the term “extended-release” refers to a dosage in which the timing and/or rate of release of a drug substance(s) is extended or lengthened at a constant rate compared to that of a conventional, immediate release dosage form. For example, the timing and/or rate of release of a drug substance can be lengthened to take place over several hours (e.g., 6-16 hours) or even over a day (e.g., 6-24 hours).

As used herein, the terms “sustained-release” and “prolonged-release” are used interchangeably and refer to a dosage form in which the timing and/or rate of release of a drug substance(s) is prolonged or lengthened as compared to that of a conventional, immediate release, dosage form. Sustained or prolonged release is characterized by a slow rise in drug concentrations in the blood, followed by a slow decline that provides therapeutic amounts long enough to reduce the frequency of administration compared with an immediate release dosage form.

As used herein, the term “controlled-release” or “CR” refers to a dosage form in which the timing and/or rate of release of a drug substance(s) is modified to allow for release of the drug substance at a specific rate such that drug levels are kept constant for a specific period of time.

As used herein, the term “delayed-release” or “DR” refers to a dosage form in which the timing and/or rate of release of a drug substance(s) is modified such that the dosage form does not release the drug substance(s) promptly after administration.

As used herein, the term “prodrug” refers to a modified variation of a parent drug that is generally biologically inactive at its site of action, but may be degraded, modified, rearranged, disassociated, or cleaved by one or more enzymatic, non-enzymatic, or other in vivo or ex vivo processes to its parent bioactive form or a derivative thereof, wherein the derivative generally maintains a bioactive component of the parent drug or a derivative thereof. Generally, a prodrug has a different pharmacokinetic profile than its parent drug such that it has, for example, improved salt formation or solubility and/or it has better systemic stability (e.g., an increased plasma half-life). A discussion of prodrugs is provided in (a) Stella, V. J.; Borchardt, R. T.; Hageman, M. J.; Oliyai, R.; Maag, H. et al. Prodrugs: Challenges and Rewards Part 1 and Part 2; Springer, p. 726: New York, N.Y., USA, 2007, (b) Rautio, J.; Kumpulainen, H.; Heimbach, T.; Oliyai, R., Oh, D. et al. Prodrugs: design and clinical applications. Nat. Rev. Drug Discov. 2008, 7, 255, (c) T. Higuchi and V. Stella, Pro-drugs as Novel Delivery Systems (1987) 14 of the A.C.S. Symposium Series, and in (d) Bioreversible Carriers in Drug Design, (1987) Edward B. Roche, ed., American Pharmaceutical Association and Pergamon Press.

As used herein, the phrase “fixed dose combination” refers to a dosage form in which two or more active drugs (e.g., a MAOI and a beta blocker) are combined in a single dosage form.

As used herein, the term “dissolution period” refers to the time required until either 80% of the drug is released from the dosage form to form a solution or an asymptote is reached in vitro. The in vitro dissolution profiles in water of dosage forms are determined according to the USP <711>.

As used herein, the term “absorption period” refers to the time required for the translocation of a drug from the site of administration into the blood stream. Thus, for example, the absorption period can be the time required until either 80% of the drug is released from the dosage form or an asymptote is reached in vivo.

As used herein, the terms “norepinephrine reuptake inhibitor” and “NRI” refer to a compound that inhibits the neurotransmitters norepinephrine (i.e., noradrenaline) and epinephrine (i.e., adrenaline) by blocking the action of the norepinephrine transporter. Examples of norepinephrine reuptake inhibitors include, but are not limited to, reboxetine, atomoxetine, viloxazine, buproprion, desipramine, maprotiline, nortriptyline, protriptyline, tapentadol, and teniloxazine.

As used herein, the term “sub-therapeutic amount” means a dose of a drug that does not achieve a particular therapeutic effect. Although this is generally not desired, drugs intended for one purpose may be administered in subtherapeutic doses to achieve a different effect. For example, a sub-therapeutic amount of a NRI can refer to a dose of the NRI that is too low to treat a disease (e.g., depression, ADHD, narcolepsy), but sufficient for another purpose (e.g., mitigating the pressor effect).

As used herein, the term “half-life” refers to the time period in which the concentration of a drug in the plasma is reduced by 50%. For example, among MAOI's, the half-life of TCP is approximately 2.5 hours, the half-life of phenelzine is approximately 11.6 hours, and the half-life of selegiline is approximately 10 hours. Among beta blockers, the half-life of Esmolol is approximately 9 minutes, the half-life of Bisoprolol ranges from 9-12 hours, and the half-life of pindolol is about 3-4 hours.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, and amides, salts of esters or amides, and N-oxides of a parent compound.

The compounds according to this disclosure may form prodrugs at hydroxyl or amino functionalities using alkoxy, amino acids, etc., groups as the prodrug forming moieties. For instance, the hydroxymethyl position may form mono-, di- or triphosphates and again these phosphates can form prodrugs. Preparations of such prodrug derivatives are discussed in various literature sources (examples are: Alexander et al., J. Med. Chem. 1988, 31, 318; Aligas-Martin et al., PCT WO 2000/041531, p. 30). The nitrogen function converted in preparing these derivatives is one (or more) of the nitrogen atoms of a compound of the disclosure.

Compounds described herein comprise atoms in both their natural isotopic abundance and in non-natural abundance. The disclosed compounds can be isotopically-labeled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³⁵S, ¹⁸F, and ³⁶Cl, respectively. Compounds further comprise prodrugs thereof, and pharmaceutically acceptable salts of said compounds or of said prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non-isotopically labeled reagent.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Strem Chemicals (Newburyport, MA), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and supplemental volumes (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### B. DOSAGE FORMS COMPRISING A MAOI AND A BETA BLOCKER

In one aspect, disclosed are dosage forms comprising: (a) a therapeutically effective amount of a monoamine oxidase inhibitor (MAOI); and (b) a therapeutically effective amount of a beta blocker selected from propranolol, metoprolol, carvedilol, timolol, carteolol, atenolol, nebivolol, sotalol, bucindolol, nadolol, celiprolol, nebivolol, betaxolol, esmolol, bisoprolol, oxprenolol, penbutolol, labetalol, acebutolol, and pindolol, or a pharmaceutically acceptable salt or free base thereof, and a pharmaceutically acceptable carrier.

In one aspect, disclosed are dosage forms comprising: (a) a therapeutically effective amount of phenelzine or a pharmaceutically acceptable salt or free base thereof; and (b) a therapeutically effective amount of a beta blocker selected from pindolol and bisoprolol, or a pharmaceutically acceptable salt or free base thereof, and a pharmaceutically acceptable carrier, wherein the MAOI is formulated as a modified-release dosage form, and wherein the beta blocker is formulated as a modified-release dosage form.

In one aspect, disclosed are dosage forms comprising a therapeutically effective amount of a MAOI, the improvement comprising further including therapeutically effective amount of a beta blocker selected from propranolol, metoprolol, carvedilol, timolol, carteolol, atenolol, nebivolol, sotalol, bucindolol, nadolol, celiprolol, nebivolol, betaxolol, esmolol, bisoprolol, oxprenolol, penbutolol, labetalol, acebutolol, and pindolol, or a pharmaceutically acceptable salt or free base thereof, in the dosage form.

In one aspect, disclosed are dosage forms comprising a therapeutically effective amount of a beta blocker, the improvement comprising further including a therapeutically effective amount of MAOI in the dosage form.

Pharmaceutically acceptable salts of the compounds are conventional acid-addition salts or base-addition salts that retain the biological effectiveness and properties of the compounds and are formed from suitable non-toxic organic or inorganic acids or organic or inorganic bases. Exemplary acid-addition salts include those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Example base-addition salts include those derived from ammonium, potassium, sodium and, quaternary ammonium hydroxides, such as for example, tetramethylammonium hydroxide. Chemical modification of a pharmaceutical compound into a salt is a known technique to obtain improved physical and chemical stability, hygroscopicity, flowability, and solubility of compounds. See, e.g., H. Ansel et. al., Pharmaceutical Dosage Forms and Drug Delivery Systems (6th Ed. 1995) at pp. 196 and 1456-1457.

In some aspects, to prolong the effect of a compound utilized herein (e.g., a MAOI or a beta blocker), it may be desirable to slow the absorption of the compound (e.g., via preparation of a modified-release formulation). This may be accomplished, for example, via a liquid suspension of crystalline or amorphous material with poor water solubility. The rate of absorption of the compound then depends upon its rate of dissolution that, in turn, may depend upon crystal size and crystalline form. Alternatively, delayed absorption of a compound can be accomplished by dissolving or suspending the compound in an oil vehicle. The modified-release dosage form can consist of micro particulates, pellets or granulation coated or uncoated in tablet, capsule, suppository, or a suspension. Modified-release formulations can also be prepared by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Alternatively, modified-release formulations can be prepared by entrapping the compound in liposomes or microemulsions that are compatible with body tissues.

Inactive ingredients for tablet, pellet, or spheres for matrix release include binding agents for solid oral dosage forms (tablets and capsules) and include pregelatinized maze starch or hydroxypropyl methylcellulose of varying degrees of substitutions; fillers (e.g. lactose, microcrystalline cellulose, calcium carbonate or calcium phosphate); disintegrants (potato starch, croscarmellose sodium, or sodium starch glycolate); wetting agents (e.g. sodium lauryl sulphate or non-ionic surfactants) or other agents suitable for tableting. In addition ingredients can include nonionic homopolymers of ethylene oxide, water soluble natural gums of polysaccharides, water swellable but insoluble, high molecular weight homopolymers and copolymers of acrylic acid chemically crosslinked with polyalkenyl alcohols, polyvinyl acetate and povidones, crosslinked amylose starch and ionic methacrylate copolymers. Additionally fatty acids, fatty acid esters and mono-, di-, tri-glycerides of differing melting points as well as hydrophobic polymers, natural occurring waxes, and ammoniomethacrylate copolymers can be used in non-swellable matrix. Lipid or hydrophobic matrices can delay the release of the drug and can be used in the delayed-release technology.

Tablets can be compressed as single, bi-layer, or multi-layer and may be coated or uncoated. Tablet coating can be functional for the release profile. Drug release can be achieved through other methods such as reservoir polymeric systems or osmotic pump systems with laser drilled tablets or capsules. Tableting methodology has many options of compressing a core tablet within an outer tablet or multiple layer tablets. The compression of the granulation or powder drug matrix is achieved through controlled pressure and appropriate dyes for the tablet formation. Pellets, granulation, or prepared spheres can be delivered to capsule shells which are designed to be immediate or modified-release.

Coating materials for modified-release of drug from formulated matrices (tablets, pellets, or spheres), which includes enteric and other release targets include shellac and zein of natural sources, derivatives of cellulose (cellulose acetate phthalate, hydroxypropyl methylcellulose phthalate) and methacrylic (acid and ester copolymers containing carboxylic functional groups) that resist solubility low pH and protect the delivery to other pH greater than 5.5. Plasticizers (such as triacetin, triethanol citrate, glycerin, and polyethylene glycol) may or may not be needed to facilitate the release profile and flexibility of the coating.

Materials for reservoir systems use the ingredients for drug release coatings surrounding the drug core matrix (most commonly a tablet) to form a controlled barrier, which may be a water insoluble coating such as cellulose acetate. The core of the tablet can contain osmotic agents such as sodium chloride and other hydrophilic polymers to push the drug out of the laser drilled orifice as designed.

Methods of manufacturing the dosage forms include the following: drug matrix preparation includes the intimate mixture of the drug and excipients to influence the drug behavior in the manufacture and drug delivery in the dosage form; granulation, compacting, hot melt and cold melt pelletizing and spherization. The resulting matrix (granulation, blend, pellet or sphere) can be a ready dosage form with tableting or encapsulation or further manipulated for the desired release profile.

Granulation of the drug matrix can be compressed/tableted as a single layer or multiple layer tablet. This tablet can be coated with coating for further control of the drug release from the tablet core. Coating can be functional for modified-release of the core or contain additional drug product for release in a multi-modal profile. Granulation or drug matrix can be further manipulated to form small pellets or spheres with methodology such as extrusion spheronization and rotary processing. Extrusion and pelletizing drug matrices are methodologies used for lipid nanoparticles and pellets.

In addition, spraying of drug substance onto inert or drug matrix spheres followed by coating in equipment designed to handle small beads/pellets/spheres. This drug loaded sphere can be further coated to deliver a controlled/modified release.

In some aspects, the disclosed modified-release dosage form is formulated such that one or more of the dissolution, release, delivery, and/or pharmacokinetic properties disclosed herein are satisfied. Thus, for example, Cmax (i.e., peak drug concentrations in blood or plasma after dosing) can be influenced by drug dose (e.g., a higher dose usually produces higher Cmax values), route of administration (e.g., higher Cmax values may occur after IV bolus dosing compared with oral dosing), and the type of formulation (e.g., a higher Cmax may occur after dosing with an immediate release oral formulation compared with a controlled-release formulation). Other drug characteristics such as solubility, permeability, ways in which it is absorbed into the body, metabolism, and metabolic products, etc., can also influence Cmax, which means that although certain projections may be made based on the factors mentioned above, the actual behavior observed is difficult to predict without significant experimentation in humans and may be unexpected. In some aspects, the disclosed modified-release dosage form has a Cmax of from about 1 ng/ml to about 500 mg/mL, 1 ng/mL to about 400 ng/mL, from about 1 ng/ml to about 300 ng/ml, from about 1 ng/mL to about 100 ng/ml, from about 1 ng/mL to about 50 ng/mL, from about 1 ng/mL to about 25 ng/ml, from about 1 ng/ml to about 10 ng/mL, from about 1 ng/mL to about 5 ng/mL, from about 5 ng/ml to 500 ng/ml, from about 10 ng/ml to about 500 ng/mL, from about 25 ng/mL to about 500 ng/mL, from about 50 ng/ml to about 500 ng/mL, from about 100 ng/ml to about 500 ng/ml from about 200 ng/ml to about 500 ng/mL, from about 300 ng/mL to about 500 ng/mL, from about 400 ng/ml to about 500 ng/ml, or from about 5 ng/mL to about 100 ng/mL after administration of a single dose. As would be appreciated by one of skill, the Cmax can be dependent on the MAOI or beta blocker being used. Thus, in various aspects, the MAOI is phenelzine and the modified-release dosage form has a Cmax of from about 5 ng/ml to about to about 250 ng/mL. In various further aspects, the beta blocker is pindolol and the modified-release dosage form has a Cmax of from about 5 ng/mL to about 100 ng/ml.

In some aspects, the disclosed modified-release dosage form has a Cmax of from about 1 ng/ml to about 500 mg/mL, 1 ng/ml to about 400 ng/mL, from about 1 ng/ml to about 300 ng/mL, from about 1 ng/mL to about 100 ng/ml, from about 1 ng/mL to about 50 ng/ml, from about 1 ng/mL to about 25 ng/ml, from about 1 ng/mL to about 10 ng/ml, from about 1 ng/ml to about 5 ng/ml, from about 5 ng/mL to 500 ng/ml, from about 10 ng/ml to about 500 ng/mL, from about 25 ng/ml to about 500 ng/ml, from about 50 ng/mL to about 500 ng/ml, from about 100 ng/ml to about 500 ng/ml from about 200 ng/ml to about 500 ng/ml, from about 300 ng/mL to about 500 ng/mL, from about 400 ng/mL to about 500 ng/mL, or from about 5 ng/ml to about 100 ng/ml after administration of 5 doses every 12 hours or at steady-state.

Tmax refers to the time at which peak drug concentration (Tmax) occurs. In some aspects, the disclosed modified-release dosage form have a Tmax of the active agent of at least about 0.5 hours, at least about 1 hour, at least about 1.5 hours, at least about 2 hours, at least about 2.5 hours, at least about 3 hours, at least about 3.5 hours, at least about 4 hours, at least about 4.5 hours, at least about 6 hours, at least about 8 hours, at least about 10 hours, at least about 11 hours, or at least about 12 hours. In some aspects, the disclosed modified-release formulations have a mean Tmax of the active agent of from about 1 hours to about 8 hours, from about 2 hours to about 6 hours, from about 3 hours to about 5 hours, or from about 1.5 hours to about 3.5 hours. As would be appreciated by one of skill, the Tmax can be dependent on the MAOI or beta blocker being used. Thus, in various aspects, the MAOI is phenelzine and the modified-release formulation has a mean Tmax of phenelzine of from about 2 hours to about 6 hours. In various further aspects, the beta blocker is pindolol and the modified-release formulation has a mean Tmax of pindolol of from about 2 hours to about 6 hours.

The term AUC means the area under the drug concentration-time curve in blood or plasma. Without wishing to be bound by theory, the AUC reflects the total body exposure to drug after dosing. Again, the size of AUC is influenced by several factors-what dose is administered, the ease and speed of drug absorption, how widely the drug is distributed in the body, and the rate of drug elimination from the body. All of these variables make it difficult to predict AUC accurately without significant experimentation in humans. In some aspects, the disclosed modified-release dosage form have a AUC 0-∞ of from about 10 ng·h/mL to about 2,000 ng·h/mL, from about 93 ng·h/mL to about 460 ng·h/mL, from about 292 ng·h/mL to about 521 ng·h/mL, from about 549 ng·h/mL to about 1543 ng·h/mL, from about 1353 ng·h/mL to about 3260 ng·h/mL, or from about 3205 ng·h/mL to about 5216 ng·h/mL after administration of a single dose. In some aspects, the disclosed modified-release dosage form have a mean AUC 0-∞ from about 3205 ng·h/mL to about 5216 ng·h/mL, from about 35 ng·h/mL to about 156 ng·h/mL, from about 58 ng·h/mL to about 287 ng·h/mL, from about 145 ng·h/mL to about 328 ng·h/mL, from about 608 ng·h/mL to about 1583 ng·h/mL, from about 1124 ng·h/mL to about 2557 ng·h/mL, or from about 2381 ng·h/mL to about 3666 ng·h/mL after administration of 5 doses every 12 hours. As would be appreciated by one of skill, the AUC can be dependent on the MAOI or beta blocker being used. Thus, in various aspects, the MAOI is phenelzine and the modified-release dosage form has an AUC 0-00 of from about 500 ng·h/mL to about 2,000 ng·h/mL. In various further aspects, the beta blocker is pindolol and the modified-release dosage form has a AUC 0-∞ of from about 50 ng·h/mL to about 800 ng·h/mL.

In some aspects, the disclosed modified-release dosage forms are formulated in accordance with routine procedures as a composition adapted for oral administration to human subjects. Compositions for oral delivery can take a variety of forms including, but not limited to, tablets, lozenges, aqueous or oil suspensions, granules, powders, emulsions, capsules, syrups, or elixirs. Orally administered compositions can also contain one or more sweetening agents such as fructose, aspartame, or saccharin; flavoring agents such as peppermint, oil of wintergreen, or cherry; coloring agents; and/or preserving agents, to provide a pharmaceutically palatable preparation. Moreover, where in tablet or pill form, the composition can be coated to delay disintegration and absorption in the gastrointestinal tract, thereby providing a sustained action over an extended period of time. Selectively permeable membranes surrounding an osmotically active compound are also suitable for oral administration. In these latter forms, fluid from the environment surrounding the capsule is imbibed by the driving compound, which swells to displace the agent or agent formulations. A time-delay material such as glycerol monostearate or glycerol stearate can also be useful. Oral compositions can include standard excipients such as mannitol, lactose, starch, magnesium stearate, sodium saccharin, cellulose, and magnesium carbonate. In some aspects, the excipients are of pharmaceutical grade.

In some aspects, the modified-release formulations can be administered by modified-release means or by delivery devices that are well known to those of ordinary skill in the art. Examples include, but are not limited to, those described in U.S. Pat. Nos. 3,845,770; 3,916,899, 3,536,809; 3,598,123; 4,008,719; 5,674,533; 5,059,595; 5,591,767; 5,120,548; 5,073,543; 5,639,476; 5,354,556; and 5,733,556. Such dosage forms can be useful for providing controlled- or sustained-release of the compositions disclosed herein using, for example, hydroxypropylmethyl cellulose, other polymer matrices, gels, permeable membranes, osmotic systems, multilayer coatings, microparticles, liposomes, microspheres, or a combination thereof to provide the desired release profile in varying proportions. Thus, in some aspects, disclosed herein are single unit dosage forms suitable for oral administration such as, but not limited to, tablets, capsules, gel caps, and caplets that are adapted for controlled- or sustained-release.

In various aspects, the dosage form consists essentially of the MAOI and the beta blocker. In a further aspect, the dosage form consists of the MAOI and the beta blocker.

In various aspects, the dosage form consists essentially of phenelzine and the beta blocker. In a further aspect, the dosage form consists of phenelzine and the beta blocker.

In various aspects, the dosage form does not contain an alpha blocker selected from doxazosin, silodosin, prazosin, tamsulosin, alfuzosin, terazosin, trimazosin, phenoxybenzamine, and phentolamine. In a further aspect, the dosage form does not contain an alpha blocker.

In various aspects, the dosage form does not contain carvedilol or labetalol.

In various aspects, the dosage form does not contain a 5HT1A agonist.

In various aspects, the MAOI is phenelzine (e.g., phenelzine sulfate) and the beta blocker is selected from propranolol, metoprolol, carvedilol, timolol, carteolol, atenolol, nebivolol, sotalol, bucindolol, nadolol, celiprolol, nebivolol, betaxolol, esmolol, bisoprolol, oxprenolol, penbutolol, labetalol, acebutolol, and pindolol, or a pharmaceutically acceptable salt or free base thereof. In a further aspect, the MAOI is phenelzine sulfate and the beta blocker is selected from pindolol and bisoprolol (e.g., bisoprolol fumarate). In a still further aspect, the MAOI is phenelzine sulfate and the beta blocker is pindolol. In a still further aspect, the MAOI is phenelzine sulfate and the beta blocker is bisoprolol fumarate.

In various aspects, the ratio of the MAOI to the beta blocker can be from about 1:1 to about 400:1, from about 2:1 to about 400:1, from about 4:1 to about 400:1, from about 8:1 to about 400:1, from about 10:1 to about 400:1, from about 20:1 to about 400:1, from about 50:1 to about 400:1, from about 100:1 to about 400:1, from about 200:1 to about 400:1, from about 1:1 to about 200:1, from about 1:1 to about 100:1, from about 1:1 to about 50:1, from about 1:1 to about 20:1, from about 1:1 to about 10:1, from about 1:1 to about 5:1, or from about 1:1 to about 2:1.

As would be appreciated by one of skill in the art, the ratio of the MAOI to the beta blocker can be dependent on the MAOI and the beta blocker being used. Thus, in various aspects, the MAOI is phenelzine and the beta blocker is pindolol, and the ratio of the MAOI to the beta blocker can be from about 1:1 to about 100:1, from about 2:1 to about 100:1, from about 4:1 to about 100:1, from about 8:1 to about 100:1, from about 10:1 to about 100:1, from about 24:1 to about 100:1, from about 50:1 to about 100:1, from about 1:1 to about 50:1, from about 1:1 to about 24:1, from about 1:1 to about 10:1, from about 1:1 to about 8:1, from about 1:1 to about 4:1, from about 1:1 to about 2:1. In various further aspects, the MAOI is phenelzine and the beta blocker is pindolol, and the ratio of the MAOI to the beta blocker is of from about 2:1 to about 24:1.

In various aspects, the MAOI and the beta blocker are co-formulated. In a further aspect, the MAOI and the beta blocker are not co-formulated.

### 1. Monoamine Oxidase Inhibitors (MAOIS)

In one aspect, the disclosed dosage forms comprise a therapeutically effective amount of a monoamine oxidase inhibitor (MAOI). Exemplary MAOIs include, but are not limited to, tranylcypromine (TCP) (e.g., tranylcypromine sulfate, tranylcypromine hydrochloride), phenelzine (e.g., phenelzine sulfate), isocarboxazid, selegiline (e.g., selegiline hydrochloride), rasagiline (e.g., rasagiline mesylate), and moclobemide (e.g., moclobemide hydrochloride).

In various aspects, the MAOI is formulated as a modified-release dosage form. In a further aspect, the MAOI is formulated as an extended-release, a sustained-release, a prolonged-release, a controlled-release, or a delayed-release dosage form. In a still further aspect, the MAOI is formulated as extended-release dosage form. In yet a further aspect, the MAOI is formulated as a sustained-release dosage form. In an even further aspect, the MAOI is formulated as a prolonged-release dosage form. In a still further aspect, the MAOI is formulated as a controlled-release dosage form. In yet a further aspect, the MAOI is formulated as a delayed-release dosage form.

In various aspects, the modified-release dosage form releases the MAOI for a dissolution period lasting from about 6 hours to about 16 hours. In a further aspect, the modified-release dosage form releases the MAOI for a dissolution period lasting from about 6 hours to about 16 hours, from about 8 hours to about 16 hours, from about 10 hours to about 16 hours, from about 12 hours to about 16 hours, from about 14 hours to about 16 hours, from about 6 hours to about 14 hours, from about 6 hours to about 12 hours, from about 6 hours to about 10 hours, from about 6 hours to about 10 hours, or from about 6 hours to about 8 hours.

In various aspects, the modified-release dosage form releases the MAOI for an absorption period lasting from about 6 hours to about 16 hours subsequent to administration to a patient. In a further aspect, the modified-release dosage form releases the MAOI for an absorption period lasting from about 6 hours to about 16 hours, from about 8 hours to about 16 hours, from about 10 hours to about 16 hours, from about 12 hours to about 16 hours, from about 14 hours to about 16 hours, from about 6 hours to about 14 hours, from about 6 hours to about 12 hours, from about 6 hours to about 10 hours, from about 6 hours to about 10 hours, or from about 6 hours to about 8 hours subsequent to administration to a patient.

In various aspects, substantially all of the MAOI is released after a time period of from about 6 hours to about 16 hours, from about 8 hours to about 16 hours, from about 10 hours to about 16 hours, from about 12 hours to about 16 hours, from about 14 hours to about 16 hours, from about 8 hours to about 14 hours, from about 8 hours to about 12 hours, from about 8 hours to about 10 hours, from about 10 hours to about 16 hours, or from about 12 hours to about 16 hours subsequent to administration to a patient.

In various aspects, the MAOI is present in an amount of from about 20 mg to about 1000 mg, from about 50 mg to about 1000 mg, from about 100 mg to about 1000 mg, from about 250 mg to about 1000 mg, from about 500 mg to about 1000 mg, from about 750 mg to about 1000 mg, from about 20 mg to about 750 mg, from about 20 mg to about 500 mg, from about 20 mg to about 250 mg, from about 20 mg to about 100 mg, from about 20 mg to about 50 mg, from about 30 mg to about 90 mg, or from about 30 mg to about 120 mg. As would be appreciated by one of skill, the amount of the MAOI present can be dependent on the MAOI being used. Thus, for example, in various aspects, the MAOI is phenelzine (e.g., phenelzine sulfate) and the MAOI is present in an amount from about 30 mg to about 120 mg.

In various aspects, the MAOI is phenelzine sulfate, and the phenelzine sulfate is present in an amount from about 20 mg to about 1000 mg, from about 50 mg to about 1000 mg, from about 100 mg to about 1000 mg, from about 250 mg to about 1000 mg, from about 500 mg to about 1000 mg, from about 750 mg to about 1000 mg, from about 20 mg to about 750 mg, from about 20 mg to about 500 mg, from about 20 mg to about 250 mg, from about 20 mg to about 100 mg, from about 20 mg to about 50 mg, from about 30 mg to about 90 mg, or from about 30 mg to about 120 mg. In some aspects, the phenelzine sulfate is present in an amount of about 30 mg to 120 mg.

In various aspects, the MAOI is selected from isocarboxazid, phenelzine, selegiline, TCP, and moclobemide. In a further aspect the MAOI is selected from isocarboxazid, phenelzine, selegiline, and TCP. In a still further aspect, the MAOI is selected from phenelzine and TCP. In yet a further aspect, the MAOI is TCP. In an even further aspect, the MAOI is isocarboxazid. In a still further aspect, the MAOI is selegiline. In yet a further aspect, the MAOI is phenelzine. In an even further aspect, the phenelzine is phenelzine sulfate.

### 2. Beta Blockers

In one aspect, the disclosed dosage forms comprise a therapeutically effective amount of a beta blocker. Exemplary beta blockers include, but are not limited to, propranolol, metoprolol, carvedilol, timolol, carteolol, atenolol, nebivolol, sotalol, bucindolol, nadolol, celiprolol, nebivolol, betaxolol, esmolol, bisoprolol, oxprenolol, penbutolol, labetalol, acebutolol, bopindolol, and pindolol, or a pharmaceutically acceptable salt or free base thereof.

In various aspects, the beta blocker is a prodrug such that the prodrug can be converted intracellularly (in vivo or in vitro) to the corresponding beta blocker. The conversion can occur by one or more mechanisms, e.g., an enzyme-catalyzed chemical reaction, a metabolic chemical reaction, and/or a spontaneous chemical reaction (e.g., solvolysis), such as, for example, through hydrolysis in blood. Examples of modifications of a parent drug to yield a prodrug include, but are not limited to: (1) ester or amide derivatives that are susceptible to being cleaved by esterases or lipases; (2) peptide derivatives that can be recognized by specific or nonspecific proteases; and (3) derivatives that cause the prodrug to accumulate at a site of action through membrane selection; and combinations of the above techniques. Prodrugs of beta blockers are commercially available or prepared by methods known to those in the art (Buur, A., et al., (1988) Intl. J. Pharmaceutics, 42 1-3, 51-60; Ghosh B., et al., (2010) Drug Delivery, 17:7, 532-540).

In various aspects, the beta blocker is pindolol or a prodrug thereof. Exemplary pindolol prodrugs include, but are not limited to, pindolol O-acetyl ester, pindolol O-propionyl ester, pindolol O-butryrl ester, pindolol O-pivaloyl ester, pindolol O-benzoyl ester, and pindolol O-cyclopropanyl ester. In various further aspects, the pindolol prodrug is a pendant chain polymeric prodrug. See, e.g., Chau et al. (1991) Drug Development and Industrial Pharmacy 17(10): 1279-1292.

In various aspects, the beta blocker is propranolol or a prodrug thereof. Exemplary propranolol prodrugs include, but are not limited to, propranolol O-acetyl ester, propranolol O-propionyl ester, propranolol O-butryrl ester, propranolol O-pivaloyl ester, propranolol O-benzoyl ester, propranolol O-cyclopropanyl ester, and a propranolol 2-oxazolidone derivative. In a further aspect, the propranolol prodrug is an O-acetyl ester, a propionyl ester, a butyryl ester, or a pivaloyl ester of propranolol. See, e.g., Buur et al. (1988) International Journal of Pharmaceutics 42(1-3): 51-60. See also D'Emanuele et al. (2004) J Control Release 95(3): 447-53. In a still further aspect, the propranolol prodrug is an ester prodrug of propranolol. See, e.g., Shameem et al. (1993) Journal of Pharmacy and Pharmacology 45(4): 246-252.

In various aspects, the beta blocker is metoprolol or a prodrug thereof. Exemplary metoprolol prodrugs include, but are not limited to, metoprolol O-acetyl ester, metoprolol O-propionyl ester, metoprolol O-butryrl ester, metoprolol O-pivaloyl ester, metoprolol O-benzoyl ester, and metoprolol O-cyclopropanyl ester. In a further aspect, the metoprolol prodrug is an acetyl ester, acetamide, or benzamide prodrug. See, e.g., Patel et al. (2016) International Journal for Pharmaceutical Research Scholars 5(3): 12-20. In a still further aspect, the metoprolol prodrug is metoprolol acetate or metoprolol propionate. See, e.g., Nair et al. (2006) Acta Pharmaceutica Sciencia 48:179-193.

In various aspects, the beta blocker is labetalol or a prodrug thereof. Exemplary labetalol prodrugs include, but are not limited to, labetalol O-acetyl ester, labetalol O-proprionyl ester, labetalol O-butryrl ester, labetalol O-pivaloyl ester, labetalol O-benzoyl ester, and labetalol O-cyclopropanyl ester.

In various aspects, the beta blocker is esmolol or a prodrug thereof. Exemplary esmolol prodrugs include, but are not limited to, esmolol O-acetyl ester, esmolol O-proprionyl ester, esmolol O-butryrl ester, esmolol O-pivaloyl ester, esmolol O-benzoyl ester, and esmolol O-cyclopropanyl ester. In a further aspect, the esmolol prodrug is selected from esmolol acetate, esmolol propionate, esmolol butyrate, and esmolol valerate. See also Bijaya et al. (2010) Drug Deliv. 17(7): 532-540.

In various aspects, the beta blocker is acebutolol or a prodrug thereof. Exemplary acebutolol prodrugs include, but are not limited to, acebutolol O-acetyl ester, acebutolol O-propionyl ester, acebutolol O-butryrl ester, acebutolol O-pivaloyl ester, acebutolol O-benzoyl ester, and acebutolol O-cyclopropanyl ester. In a further aspect, the acebutolol prodrug is an O-cyclopropane carboxylic acid ester. See, e.g., Hovgaard et al. (1995) Pharm. Res. 12(3): 387-92.

In various aspects, the beta blocker is a timolol or a prodrug thereof. Exemplary timolol prodrugs include, but are not limited to timolol O-acetyl ester, timolol O-proprionyl ester, timolol O-butryrl ester, timolol O-pivaloyl ester, timolol O-benzoyl ester, and timolol O-cyclopropanyl ester. See e.g., Chang et al. (1987) Invest Ophthalmol Vis Sci. March 28(3): 487-91. In various further aspects, the timolol prodrug is an amphiphilic timolol prodrug. In a yet further aspect, the timolol prodrug is selected from octanoyl timolol, decanoyl timolol, dodecanoyl timolol, myristoyl timolol, and palmitoyl timolol. See, e.g., Pech et al. (1993) J Ocul Pharmacol. Summer 9(2): 141-50.

In various aspects, the beta blocker is carvedilol or a prodrug thereof. Exemplary carvedilol prodrugs include, but are not limited to, carvedilol O-acetyl ester, carvedilol O-propionyl ester, carvedilol O-butyryl ester, carvedilol O-pivaloyl ester, carvedilol O-benzoyl ester, and carvedilol O-cyclopropanyl ester.

In various aspects, the beta blocker is atenolol or a prodrug thereof. Exemplary atenolol prodrugs include, but are not limited to, atenolol O-acetyl ester, atenolol O-propionyl ester, atenolol O-butyryl ester, atenolol O-pivaloyl ester, atenolol O-benzoyl ester, and atenolol O-cyclopropanyl ester. In a further aspect, the atenolol prodrug is atenolol N-maleic amide or atenolol N-(methyl)maleic amide. See, e.g., Karaman et al. (2014) Scientific World Journal January 12:248651.

In various aspects, the beta blocker is nadolol or a prodrug thereof. Exemplary nadolol prodrugs include, but are not limited to, nadolol O-acetyl ester, nadolol O-propionyl ester, nadolol O-butyryl ester, nadolol O-pivaloyl ester, nadolol O-benzoyl ester, and nadolol O-cyclopropanyl ester. In a further aspect, the nadolol prodrug is selected from diacetyl nadolol, dilaurate nadolol, dibenzoyl nadolol, and di(phenylacetyl) nalodionol. See e.g., U.S. Pat. No. 4,029,676 and Chiang et al. (1987) J Pharm Sci. December 76(12): 914-7.

In various aspects, the beta blocker is oxprenolol or a prodrug thereof. Exemplary oxprenolol prodrugs include, but are not limited to, oxprenolol O-acetyl ester, oxprenolol O-propionyl ester, oxprenolol O-butyryl ester, oxprenolol O-pivaloyl ester, oxprenolol O-benzoyl ester, and oxprenolol O-cyclopropanyl ester. See, e.g., Jordan (1997) J Pharm Sci. October 86(10): 1085-91 and Kour et al. (2021) Asian J Pharm Sci, March 16(2): 175-191.

In various aspects, the beta blocker is bisoprolol or a prodrug thereof. Exemplary bisoprolol prodrugs include, but are not limited to, bisoprolol O-acetyl ester, bisoprolol O-propionyl ester, bisoprolol O-butyryl ester, bisoprolol O-pivaloyl ester, bisoprolol O-benzoyl ester, and bisoprolol O-cyclopropanyl ester.

In various aspects, the beta blocker is carteolol or a prodrug thereof. Exemplary carteolol prodrugs include, but are not limited to, carteolol O-acetyl ester, carteolol O-propionyl ester, carteolol O-butyryl ester, carteolol O-pivaloyl ester, carteolol O-benzoyl ester, and carteolol O-cyclopropanyl ester.

In various aspects, the beta blocker is nebivolol or a prodrug thereof. Exemplary nebivolol prodrugs include, but are not limited to, nebivolol mono O-acetyl ester, nebivolol bis O-acetyl ester nebivolol mono O-proprionyl ester, nebivolol bis O-proprionyl ester nebivolol mono O-butryrl ester, nebivolol bis O-butryrl ester, nebivolol mono O-pivaloyl ester, nebivolol bis O-pivaloyl ester, nebivolol mono O-benzoyl ester, nebivolol bis O-benzoyl ester nebivolol mono O-cyclopropanyl ester, and nebivolol bis O-cyclopropanyl ester.

In various aspects, the beta blocker is sotalol or a prodrug thereof. Exemplary sotalol prodrugs include, but are not limited to, sotalol O-acetyl ester, sotalol O-proprionyl ester, sotalol O-butryrl ester, sotalol O-pivaloyl ester, sotalol O-benzoyl ester, and sotalol O-cyclopropanyl ester.

In various aspects, the beta blocker is bucindolol or a prodrug thereof. Exemplary bucindolol prodrugs include, but are not limited to, bucindolol O-acetyl ester, bucindolol O-propionyl ester, bucindolol O-butryrl ester, bucindolol O-pivaloyl ester, bucindolol O-benzoyl ester, and bucindolol O-cyclopropanyl ester.

In various aspects, the beta blocker is betaxolol or a prodrug thereof. Exemplary betaxolol prodrugs include, but are not limited to, betaxolol O-acetyl ester, betaxolol O-proprionyl ester, betaxolol O-butryrl ester, betaxolol O-pivaloyl ester, betaxolol O-benzoyl ester, and betaxolol O-cyclopropanyl ester.

In various aspects, the beta blocker is penbutolol or a prodrug thereof. Exemplary penbutolol prodrugs include, but are not limited to, penbutolol O-acetyl ester, penbutolol O-propionyl ester, penbutolol O-butryrl ester, penbutolol O-pivaloyl ester, penbutolol O-benzoyl ester, and penbutolol O-cyclopropanyl ester.

In various aspects, the beta blocker is formulated as a modified-release dosage form. In a further aspect, the beta blocker is formulated as an extended-release, a sustained-release, a prolonged-release, a controlled-release, or a delayed-release dosage form. In a still further aspect, the beta blocker is formulated as an extended-release dosage form. In yet a further aspect, the beta blocker is formulated as a sustained-release dosage form. In an even further aspect, the beta blocker is formulated as a prolonged-release dosage form. In a still further aspect, the beta blocker is formulated as a controlled-release dosage form. In yet a further aspect, the beta blocker is formulated as a delayed-release dosage form.

In various aspects, the modified-release dosage form releases the beta blocker for a dissolution period lasting from about 6 hours to about 24 hours. In a further aspect, the modified-release dosage form releases the beta blocker for a dissolution period lasting from about 7 hours to about 24 hours, from about 8 hours to about 24 hours, from about 10 hours to about 24 hours, from about 12 hours to about 24 hours, from about 14 hours to about 24 hours, from about 16 hours to about 24 hours, from about 18 hours to about 24 hours, from about 20 hours to about 24 hours, from about 22 hours to about 24 hours, from about 6 hours to about 22 hours, from about 6 hours to about 20 hours, from about 6 hours to about 18 hours, from about 6 hours to about 16 hours, from about 6 hours to about 14 hours, from about 6 hours to about 12 hours, from about 6 hours to about 10 hours, from about 6 hours to about 10 hours, from about 6 hours to about 8 hours, or from about 6 hours to about 7 hours. In a still further aspect, the modified-release dosage form releases the beta blocker for a dissolution period lasting from about 7 hours to about 24 hours.

In various aspects, the modified-release dosage form releases the beta blocker for an absorption period lasting from about 6 hours to about 24 hours subsequent to administration to a patient. In a further aspect, the modified-release dosage form releases the beta blocker for an absorption period lasting from about 7 hours to about 24 hours, from about 8 hours to about 24 hours, from about 10 hours to about 24 hours, from about 12 hours to about 24 hours, from about 14 hours to about 24 hours, from about 16 hours to about 24 hours, from about 18 hours to about 24 hours, from about 20 hours to about 24 hours, from about 22 hours to about 24 hours, from about 6 hours to about 22 hours, from about 6 hours to about 20 hours, from about 6 hours to about 18 hours, from about 6 hours to about 16 hours, from about 6 hours to about 14 hours, from about 6 hours to about 12 hours, from about 6 hours to about 10 hours, from about 6 hours to about 10 hours, from about 6 hours to about 8 hours, or from about 6 hours to about 7 hours subsequent to administration to a patient. In a still further aspect, the modified-release dosage form releases the beta blocker for an absorption period lasting from about 7 hours to about 24 hours subsequent to administration to a patient.

In various aspects, substantially all of the beta blocker is released after a time period of from about 6 hours to about 24 hours, from about 8 hours to about 24 hours, from about 10 hours to about 24 hours, from about 12 hours to about 24 hours, from about 14 hours to about 24 hours, from about 16 hours to about 24 hours, from about 4 hours to about 24 hours, from about 8 hours to about 24 hours, from about 8 hours to about 10 hours, from about 10 hours to about 16 hours, or from about 12 hours to about 16 hours subsequent to administration to a patient.

In various aspects, the beta blocker is formulated as a modified-release dosage form and the MAOI is formulated as a modified-release dosage form, and the MAOI and the beta blocker are released over different time periods.

In various aspects, the beta blocker is formulated as a modified-release dosage form and the MAOI is formulated as a modified-release dosage form, and wherein the MAOI and the beta blocker are released over approximately the same time periods.

In various aspects, the beta blocker can have a low half-life such as, for example, a half-life of from about 1 hour to about 6 hours, from about 2 hours to about 6 hours, from about 3 hours to about 6 hours, from about 4 hours to about 6 hours, from about 1 hour to about 5 hours, from about 1 hours to about 4 hours, from about 1 hours to 3 hours, from 1 hours to 2 hours, from about 2 hour to about 5 hours, from about 2 hours to about 4 hours, from about 2 hours to about 3 hours, from about 3 hours to about 5 hours, or from about 3 hours to about 4 hours. In a further aspect, the half-life is from about 3 hours to about 4 hours.

In various aspects, the beta blocker can have a low Kᵢ at beta receptors such as, for example, a Kᵢ of from about 1 nM to about 500 nM, from about 5 nM to about 500 nM, from about 10 nM to about 500 nM, from about 20 nM to about 500 nM, from about 30 nM to about 500 nM, from about 40 nM to about 500 nM, from about 50 nM to about 500 nM, from about 100 nM to about 500 nM, from about 200 nM to about 500 nM, from about 300 nM to about 500 nM, from about 400 nM to about 500 nM, from about 1 nM to about 400 nM, from about 1 nM to about 300 nM, from about 1 nM to about 200 nM, from about 1 nM to about 100 nM, from about 1 nM to about 50 nM, from about 1 nM to about 40 nM, from about 1 nM to about 30 nM, from about 1 nM to about 20 nM, from about 1 nM to about 10 nM, from about 1 nM to about 5 nM, from about 5 nM to about 10 nM, from about 10 nM to about 20 nM, from about 20 nM to about 30 nM, from about 30 nM to about 40 nM, from about 40 mM to about 50 nM, from about 50 nM to about 100 nM, from about 100 nM to about 200 nM, from about 200 nM to about 300 nM, or from about 300 nM to about 400 nM, as determined using the Hoffmann method. See Hoffman et al. (2004) Naunyn-Schmiedeberg's Arch Pharmacol 369:151-159. In various further aspects, the beta blocker can have a Kᵢ at beta receptors of less than 1 nM as determined using the Hoffmann method. As would be appreciated by one of skill, the Kᵢ of the beta blocker present can be dependent on the beta blocker being used.

In various aspects, the beta blocker can have a high Kᵢ at alpha receptors such as, for example, a Kᵢ of at least about 5,000, at least about 6,000 nM, at least about 7,000 nM, at least about 8,000 nM, or at least about 9,000 nM, as determined using the Hoffmann method. See Hoffman et al. (2004) Naunyn-Schmiedeberg's Arch Pharmacol 369:151-159. As would be appreciated by one of skill, the Kᵢ of the beta blocker present can be dependent on the beta blocker being used.

In various aspects, the beta blocker exhibits a ratio of beta:alpha blockade of at least about 10:1, at least about 100:1, at least about 500:1, at least about 1,000:1, at least about 2,000:1, or at least about 3,000:1.

In various aspects, the beta blocker can have a large therapeutic window such as, for example, a therapeutic window of from about 12 hours to about 24 hours, about 12 hours to about 20 hours, about 12 hour to about 16 hours, about 16 hours to about 24 hours, about 20 hours to about 24 hours, or about 16 hours to about 20 hours. As would be understood by one of skill in the art, a large therapeutic window can be achieved by, for example, selecting a beta blocker with a low half-life (e.g., 3-4 hours) and low Kᵢ.

In various aspects, the beta blocker is present in an amount of from about 2.5 mg to about 1000 mg. In a further aspect, the beta blocker is present in an amount of from about 2.5 mg to about 800 mg, from about 2.5 mg to about 600 mg, from about 2.5 mg to about 400 mg, from about 2.5 mg to about 200 mg, from about 2.5 mg to about 100 mg, from about 2.5 mg to about 80 mg, from about 2.5 mg to about 60 mg, from about 2.5 mg to about 40 mg, from about 2.5 mg to about 30 mg, from about 5 mg to about 1000 mg, from about 10 mg to about 1000 mg, from about 20 mg to about 100 mg, from about 200 mg to about 1000 mg, from about 400 mg to about 1000 mg, from about 600 mg to about 1000 mg, from about 800 mg to about 1000 mg, from about 5 mg to about 30 mg, from about 10 mg to about 40 mg, from about 20 mg to about 50 mg, or from about 30 mg to about 90 mg.

As would be appreciated by one of skill, the amount of the beta blocker present can be dependent on the beta blocker being used. Thus, in various aspects, the beta blocker is pindolol and the beta blocker is present in an amount of from about 2.5 mg to about 60 mg. In a yet further aspects, the beta blocker is bisoprolol (e.g., bisoprolol fumarate) and the beta blocker is present in an amount of from about 2.5 mg to about 30 mg.

In various aspects, pindolol is present in an amount of from about 2.5 mg to about 1000 mg, In some aspects, pindolol is present in an amount of from about 2.5 mg to about 800 mg, from about 2.5 mg to about 600 mg, from about 2.5 mg to about 400 mg, from about 2.5 mg to about 200 mg, from about 2.5 mg to about 100 mg, from about 2.5 mg to about 80 mg, from about 2.5 mg to about 60 mg, from about 2.5 mg to about 40 mg, from about 2.5 mg to about 30 mg, from about 5 mg to about 1000 mg, from about 10 mg to about 1000 mg, from about 20 mg to about 100 mg, from about 200 mg to about 1000 mg, from about 400 mg to about 1000 mg, from about 600 mg to about 1000 mg, from about 800 mg to about 1000 mg, from about 5 mg to about 30 mg, from about 10 mg to about 40 mg, from about 20 mg to about 50 mg, or from about 30 mg to about 90 mg. In further aspects, pindolol is present in an amount of from about 2.5 mg to about 60 mg. In a yet further aspects, pindolol is present in an amount of from about 2.5 mg to about 30 mg.

In various aspects, bisoprolol fumarate is present in an amount of from about 2.5 mg to about 1000 mg, In some aspects, bisoprolol fumarate is present in an amount of from about 2.5 mg to about 800 mg, from about 2.5 mg to about 600 mg, from about 2.5 mg to about 400 mg, from about 2.5 mg to about 200 mg, from about 2.5 mg to about 100 mg, from about 2.5 mg to about 80 mg, from about 2.5 mg to about 60 mg, from about 2.5 mg to about 40 mg, from about 2.5 mg to about 30 mg, from about 5 mg to about 1000 mg, from about 10 mg to about 1000 mg, from about 20 mg to about 100 mg, from about 200 mg to about 1000 mg, from about 400 mg to about 1000 mg, from about 600 mg to about 1000 mg, from about 800 mg to about 1000 mg, from about 5 mg to about 30 mg, from about 10 mg to about 40 mg, from about 20 mg to about 50 mg, or from about 30 mg to about 90 mg. In further aspects, bisoprolol fumarate is present in an amount of from about 2.5 mg to about 60 mg. In a yet further aspects, bisoprolol fumarate is present in an amount of from about 2.5 mg to about 30 mg.

In various aspects, the beta blocker is selected from propranolol, metoprolol, carvedilol, timolol, carteolol, atenolol, nebivolol, sotalol, bucindolol, nadolol, celiprolol, nebivolol, betaxolol, esmolol, bisoprolol, oxprenolol, penbutolol, labetalol, acebutolol, and pindolol, or a pharmaceutically acceptable salt or free base thereof. In further aspects, the beta blocker is selected from pindolol and bisoprolol, or a pharmaceutically acceptable salt or free base thereof.

In various aspects, the beta blocker is selected from oxprenolol, penbutolol, labetalol, acebutolol, and pindolol, or a pharmaceutically acceptable salt or free base thereof. In a further aspect, the beta blocker is selected from pindolol, carteolol, penbutolol, and acebutolol, or a pharmaceutically acceptable salt or free base thereof. In yet a further aspect, the beta blocker is pindolol. In yet a further aspect, the beta blocker is bisoprolol, or a pharmaceutically acceptable salt or free base thereof. In a still further aspect, the beta blocker is bisoprolol fumarate.

In various aspects, the beta blocker is a non-selective beta blocker. In a further aspect, the beta blocker is a selective beta blocker (e.g., a beta-1 selective beta blocker). In a still further aspect, the beta blocker is a beta blocker with ISA. In a still further aspect, the beta blocker is an alpha- and beta-dual blocker.

In various aspects, the beta blocker is an alpha and beta dual receptor blocker. In a further aspect, the alpha and beta dual receptor blocker is selected from labetalol and carvedilol, or a pharmaceutically acceptable salt or free base thereof. In a still further aspect, the dual alpha and beta blocker is labetalol, or a pharmaceutically acceptable salt or free base thereof. In a still further aspect, the dual alpha and beta blocker is carvedilol, or a pharmaceutically acceptable salt or free base thereof.

In various aspects, the beta blocker can have intrinsic sympathomimetic activity (ISA). In a further aspect, the beta blocker with ISA is selected from pindolol, carteolol, penbutolol, and acebutolol, or a pharmaceutically acceptable salt or free base thereof. In a further aspect, the beta blocker with ISA is selected from acebutolol and pindolol, or a pharmaceutically acceptable salt or free base thereof. In a further aspect, the beta blocker with ISA is pindolol, or a pharmaceutically acceptable salt or free base thereof. In a further aspect, the beta blocker with ISA is acebutolol, or a pharmaceutically acceptable salt or free base thereof.

In various aspects, the beta blocker is a beta-1 selective beta blocker. In a further aspect, beta-1 selective beta blocker is selected from atenolol, betaxolol, bisoprolol, esmolol, acebutolol, metoprolol, and nebivolol, or a pharmaceutically acceptable salt or free base thereof.

### 3. Norepinephrine Reuptake Inhibitors (NRIs)

In various aspects, the dosage form further comprises an effective amount of a norepinephrine reuptake inhibitor (NRI). Exemplary NRIs include, but are not limited to, reboxetine, atomoxetine, viloxazine, buproprion, desipramine, maprotiline, nortriptyline, protriptyline, tapentadol, and teniloxazine. In a further aspect, the NRI is selected from, buproprion, desipramine, maprotiline, nortriptyline, protriptyline, tapentadol, and teniloxazine. In still further aspect, the NRI is selected from nortriptyline and protriptyline. In yet a further aspect, the NRI is protriptyline.

In various aspects, the effective amount of the NRI is a therapeutic amount. In a further aspect, the effective amount of the NRI is a sub-therapeutic amount.

In various aspects, the NRI is formulated as a modified-release dosage form. Thus, in some aspects, the NRI is formulated as an extended-release, a sustained-release, a prolonged-release, a controlled-release, or a delayed-release dosage form. In a further aspect, the NRI is formulated as an extended-release dosage form. In a still further aspect, the NRI is formulated as a sustained-release dosage form. In an even further aspect, the NRI is formulated as a prolonged-release dosage form. In yet a further aspect, the NRI is formulated as a controlled-release dosage form. In a still further aspect, the NRI is formulated as a delayed-release dosage form.

In various aspects, the NRI is present in an amount of from about 1 mg to about 1000 mg. In a further aspect, the NRI is present in an amount of from about 1 mg to about 800 mg, from about 1 mg to about 600 mg, from about 1 mg to about 400 mg, from about 1 mg to about 200 mg, from about 1 mg to about 100 mg, from about 1 mg to about 80 mg, from about 1 mg to about 60 mg, from about 1 mg to about 40 mg, from about 1 mg to about 30 mg, from about 5 mg to about 1000 mg, from about 10 mg to about 1000 mg, from about 20 mg to about 100 mg, from about 200 mg to about 1000 mg, from about 400 mg to about 1000 mg, from about 600 mg to about 1000 mg, from about 800 mg to about 1000 mg, from about 5 mg to about 30 mg, from about 10 mg to about 40 mg, from about 20 mg to about 50 mg, or from about 30 mg to about 90 mg. As would be appreciated by one of skill in the art, the amount of the NRI present can be dependent on the NRI being used. Thus, in various aspects, the NRI is protriptyline and the NRI is present in an amount of from about 1 mg to about 150 mg. In a further aspect, the NRI is present in an amount of from about 5 mg to about 50 mg.

In various aspects, protriptyline is present in an amount of from about 1 mg to about 1000 mg. In a further aspect, protriptyline is present in an amount of from about 1 mg to about 800 mg, from about 1 mg to about 600 mg, from about 1 mg to about 400 mg, from about 1 mg to about 200 mg, from about 1 mg to about 100 mg, from about 1 mg to about 80 mg, from about 1 mg to about 60 mg, from about 1 mg to about 40 mg, from about 1 mg to about 30 mg, from about 5 mg to about 1000 mg, from about 10 mg to about 1000 mg, from about 20 mg to about 100 mg, from about 200 mg to about 1000 mg, from about 400 mg to about 1000 mg, from about 600 mg to about 1000 mg, from about 800 mg to about 1000 mg, from about 5 mg to about 30 mg, from about 10 mg to about 40 mg, from about 20 mg to about 50 mg, or from about 30 mg to about 90 mg. In a still further aspect, protriptyline is present in an amount of from about 1 mg to about 150 mg. In yet a further aspect, protriptyline is present in an amount of from about 5 mg to about 50 mg.

In various aspects, the ratio of the MAOI to the NRI is from about 100:1 to about 1:1. In a further aspect, the ratio of the MAOI to the NRI is from about 100:1 to about 2:1, from about 100:1 to about 5:1, from about 100:1 to about 10:1, from about 100:1 to about 20:1, from about 100:1 to about 50:1, from about 50:1 to about 1:1, from about 20:1 to about 1:1, from about 10:1 to about 1:1, from about 5:1 to about 1:1, from about 2:1 to about 1:1. As would be appreciated by one of skill, the ratio of the MAOI to the NRI can be dependent on the MAOI and NRI being used. Thus, in various aspects, the MAOI is phenelzine (e.g., 60-90 mg/day) and the NRI is protriptyline (e.g., 15-40 mg/day), and the ratio of the MAOI to the NRI is from about 10:1 to about 1:2.

In various aspects, the ratio of the beta blocker to the NRI is from about 1:1 to about 100:1. In a further aspect, the ratio of the beta blocker to the NRI is from about 2:1 to about 100:1, from about 5:1 to about 100:1, from about 10:1 to about 100:1, from about 20:1 to about 100:1, from about 50:1 to about 100:1, from about 1:1 to about 50:1, from about 1:1 to about 20:1, from about 1:1 to about 10:1, from about 1:1 to about 5:1, from about 1:1 to about 2:1. As would be appreciated by one of skill, the ratio of the beta blocker to the NRI can be dependent on the beta blocker and NRI being used. Thus, in various aspects, the beta blocker is pindolol (e.g., 5-60 mg/day) and the NRI is protriptyline (e.g., 15-40 mg/day), and the ratio of the beta blocker to the NRI is from about 3:2 to about 1:3.

### C. METHODS FOR TREATING A PSYCHIATRIC DISORDER

The invention also provides methods for treating a psychiatric disorder in a subject by administering a dosage form comprising a monoamine oxidase inhibitor (MAOI) and a beta blocker selected from propranolol, metoprolol, carvedilol, timolol, carteolol, atenolol, nebivolol, sotalol, bucindolol, nadolol, celiprolol, nebivolol, betaxolol, esmolol, bisoprolol, oxprenolol, penbutolol, labetalol, acebutolol, and pindolol, or a pharmaceutically acceptable salt or free base thereof. In various aspects, one or both of the MAOI and the beta blocker is formulated as a modified-release dosage form, such that the MAOI and the beta blocker can be released over approximately the same or different absorption periods.

Thus, in one aspect, disclosed are methods for treating a psychiatric disorder in a subject, the method comprising administering to the subject an effective amount of a disclosed dosage form.

In one aspect, disclosed are methods for treating a psychiatric disorder in a subject in need thereof, the method comprising administering to the subject: (a) a therapeutically effective amount of a monoamine oxidase inhibitor (MAOI); and (b) a therapeutically effective amount of a beta blocker selected from propranolol, metoprolol, carvedilol, sotalol, bucindolol, nadolol, celiprolol, nebivolol, betaxolol, esmolol, bisoprolol, oxprenolol, penbutolol, labetalol, acebutolol, and pindolol, or a pharmaceutically acceptable salt or free base thereof, wherein the subject has not previously been diagnosed as having migraines, and wherein the subject is not currently experiencing a migraine.

In one aspect, disclosed are methods for treating a psychiatric disorder in a subject in need thereof by administering a MAOI, the improvement comprising simultaneously administering a beta blocker selected from propranolol, metoprolol, carvedilol, timolol, carteolol, atenolol, nebivolol, sotalol, bucindolol, nadolol, celiprolol, nebivolol, betaxolol, esmolol, bisoprolol, oxprenolol, penbutolol, labetalol, acebutolol, and pindolol, or a pharmaceutically acceptable salt or free base thereof, to the patient in an amount that, together with the MAOI, is effective to reduce migraine relapse and/or produce longer lasting efficacy compared to the administration of the MAOI in the absence of the beta blocker, wherein the MAOI and the beta blocker are together present in a single dosage form.

In one aspect, disclosed are methods for treating a psychiatric disorder in a subject in need thereof by administering a MAOI, the improvement comprising simultaneously administering a beta blocker selected from propranolol, metoprolol, carvedilol, timolol, carteolol, atenolol, nebivolol, sotalol, bucindolol, nadolol, celiprolol, nebivolol, betaxolol, esmolol, bisoprolol, oxprenolol, penbutolol, labetalol, acebutolol, and pindolol, or a pharmaceutically acceptable salt or free base thereof, to the patient in an amount that, together with the MAOI, is effective to reduce migraine relapse and/or produce longer lasting efficacy compared to the administration of the MAOI in the absence of the beta blocker, wherein the subject has not previously been diagnosed as having migraines and wherein the subject is not currently experiencing a migraine.

In one aspect, disclosed are methods for treating a psychiatric disorder in a subject in need thereof by administering a beta blocker, the improvement comprising simultaneously administering a MAOI to the patient in an amount that, together with the beta blocker, is effective to reduce migraine relapse and/or produce longer lasting efficacy compared to the administration of the beta blocker in the absence of the MAOI, wherein the MAOI and the beta blocker are together present in a single dosage form.

In one aspect, disclosed are methods for treating a psychiatric disorder in a subject in need thereof by administering a beta blocker, the improvement comprising simultaneously administering a beta blocker selected from propranolol, metoprolol, carvedilol, timolol, carteolol, atenolol, nebivolol, sotalol, bucindolol, nadolol, celiprolol, nebivolol, betaxolol, esmolol, bisoprolol, oxprenolol, penbutolol, labetalol, acebutolol, and pindolol, or a pharmaceutically acceptable salt or free base thereof, to the patient in an amount that, together with the MAOI, is effective to reduce migraine relapse and/or produce longer lasting efficacy compared to the administration of the MAOI in the absence of the beta blocker, wherein the subject has not previously been diagnosed as having migraines and wherein the subject is not currently experiencing a migraine.

In one aspect, disclosed are methods for treating a psychiatric disorder in a subject in need thereof, the method comprising administering to the subject an effective amount of a dosage form comprising: (a) a therapeutically effective amount of a monoamine oxidase inhibitor (MAOI) selected from phenelzine and tranylcypromine (TCP); (b) a therapeutically effective amount of a beta blocker selected from pindolol and carvedilol, or a pharmaceutically acceptable salt or free base thereof; and (c) a pharmaceutically acceptable carrier, wherein the subject has not previously been diagnosed as having migraines.

To treat or control the disorder, the compounds and pharmaceutical compositions comprising the compounds are administered to a subject in need thereof, such as a vertebrate, e.g., a mammal, a fish, a bird, a reptile, or an amphibian. The subject can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. The subject is preferably a mammal, such as a human. Prior to administering the compounds or compositions, the subject can be diagnosed with a need for treatment of a psychiatric disorder, such as depression.

The modified-release dosage forms can be administered to the subject according to any method. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration and parenteral administration, including injectable such as intravenous administration, intra-arterial administration, intramuscular administration, and subcutaneous administration. Administration can be continuous or intermittent. A preparation can be administered therapeutically; that is, administered to treat an existing disease or condition. A preparation can also be administered prophylactically; that is, administered for prevention of an infection or condition, such as a psychiatric disorder.

The therapeutically effective amount or dosage of the compound can vary within wide limits. Such a dosage is adjusted to the individual requirements in each particular case including the specific compound(s) being administered, the route of administration, the condition being treated, as well as the patient being treated. In general, in the case of oral or parenteral administration to adult humans weighing approximately 70 Kg or more, a daily dosage of about 10 mg to about 10,000 mg, preferably from about 200 mg to about 1,000 mg, should be appropriate, although the upper limit may be exceeded. The daily dosage can be administered as a single dose or in divided doses, or for parenteral administration, as a continuous infusion. Single dose compositions can contain such amounts or submultiples thereof of the compound or composition to make up the daily dose. The dosage can be adjusted by the individual physician in the event of any contraindications. Dosage can vary, and can be administered in one or more dose administrations daily, for one or several days.

In various aspects, the psychiatric disorder is depression. Examples of depression include, but are not limited to, major depressive disorder, major depressive disorder with anxious distress, treatment resistant depression, anxious depression, and mixed anxiety and depression. In a further aspect, the depression is selected from major depressive disorder, major depressive disorder with anxious distress, and treatment resistant depression. In a yet further aspect, the depression is major depressive disorder. In a still further aspect, the depression is major depressive disorder with anxious distress. In a yet further aspect, the depression is treatment resistant depression,

In various aspects, the subject is not currently on a tyramine restricted diet. In a further aspect, the subject is not on a tyramine restricted diet subsequent to the administering step. In a still further aspect, the tyramine restricted diet precludes consumption of a meal containing 100 mg of tyramine or more. For example, the tyramine restricted diet can preclude consumption of a meal containing at least 100 mg, at least 150 mg, at least 200 mg, at least 250 mg, at least 300 mg, at least 350 mg, at least 400 mg, at least 450 mg, or at least 500 mg of tyramine.

In various aspects, the dosage form is administered at a dosage of from about 100 mg to about 1000 mg. In a further aspect, the dosage form is administered at a dosage of from about 200 mg to about 1000 mg, from about 300 mg to about 1000 mg, from about 400 mg to about 1000 mg, from about 500 mg to about 1000 mg, from about 600 mg to about 1000 mg, from about 700 mg to about 1000 mg, from about 800 mg to about 1000 mg, from about 900 mg to about 1000 mg, from about 100 mg to about 900 mg, from about 100 mg to about 800 mg, from about 100 mg to about 700 mg, from about 100 mg to about 600 mg, from about 100 mg to about 500 mg, from about 100 mg to about 400 mg, from about 100 mg to about 300 mg, from about 100 mg to about 200 mg, from about 200 mg to about 300 mg, from about 300 mg to about 400 mg, from about 400 mg to about 500 mg, from about 500 mg to about 600 mg, from about 600 mg to about 700 mg, from about 700 mg to about 800 mg, or from about 800 mg to about 900 mg. In a further aspect, the dosage form is administered at a dosage of from about 150 mg to about 500 mg.

In various aspects, the dosage form is administered once per day. In a further aspect, the dosage form is administered twice per day.

In various aspects, the MAOI and the beta blocker are administered simultaneously. In a further aspect, the MAOI and the beta blocker are administered sequentially.

In various aspects, the MAOI and the beta blocker are administered as a fixed dose combination.

In various aspects, the MAOI and the beta blocker are co-formulated. In a further aspect, the MAOI and the beta blocker are not co-formulated.

In various aspects, the MAOI is not phenelzine.

In various aspects, the method comprises administering to the subject exactly two active agents, wherein the two active agents are the MAOI and the beta blocker. In various further aspects, the method comprises administering to the subject exactly two active agents, wherein the two active agents are phenelzine and the beta blocker.

In various aspects, an alpha blocker selected from doxazosin, silodosin, prazosin, tamsulosin, alfuzosin, terazosin, trimazosin, phenoxybenzamine, and phentolamine has not been administered to the subject within a time period of about one week or less prior to or after the administering step. In a further aspect, an alpha blocker selected from doxazosin, silodosin, prazosin, tamsulosin, alfuzosin, terazosin, trimazosin, phenoxybenzamine, and phentolamine has not been administered to the subject within a time period of about twenty-four hours or less prior to or after the administering step. In a still further aspect, an alpha blocker selected from doxazosin, silodosin, prazosin, tamsulosin, alfuzosin, terazosin, trimazosin, phenoxybenzamine, and phentolamine is not simultaneously administered to the subject.

In various aspects, an alpha blocker has not been administered to the subject within a time period of about one week or less prior to or after the administering step. In a further aspect, an alpha blocker has not been administered to the subject within a time period of about twenty-four hours or less prior to or after the administering step. In a still further aspect, an alpha blocker is not simultaneously administered to the subject.

In various aspects, neither carvedilol nor labetalol has been administered to the subject within a time period of about one week or less prior to or after the administering step. In a further aspect, neither carvedilol nor labetalol has been administered to the subject within a time period of about twenty-four hours or less prior to or after the administering step. In a still further aspect, neither carvedilol nor labetalol is simultaneously administered to the subject.

In various aspects, a 5HT1A agonist has not been administered to the subject within a time period of about one week or less prior to or after the administering step. In a further aspect, a 5HT1A agonist has not been administered to the subject within a time period of about twenty-four hours or less prior to or after the administering step. In a still further aspect, a 5HT1A agonist is not simultaneously administered to the subject.

In various aspects, the beta blocker can have a low Kᵢ at beta receptors such as, for example, a Kᵢ of from about 1 nM to about 500 nM, from about 5 nM to about 500 nM, from about 10 nM to about 500 nM, from about 20 nM to about 500 nM, from about 30 nM to about 500 nM, from about 40 nM to about 500 nM, from about 50 nM to about 500 nM, from about 100 nM to about 500 nM, from about 200 nM to about 500 nM, from about 300 nM to about 500 nM, from about 400 nM to about 500 nM, from about 1 nM to about 400 nM, from about 1 nM to about 300 nM, from about 1 nM to about 200 nM, from about 1 nM to about 100 nM, from about 1 nM to about 50 nM, from about 1 nM to about 40 nM, from about 1 nM to about 30 nM, from about 1 nM to about 20 nM, from about 1 nM to about 10 nM, from about 1 nM to about 5 nM, from about 5 nM to about 10 nM, from about 10 nM to about 20 nM, from about 20 nM to about 30 nM, from about 30 nM to about 40 nM, from about 40 mM to about 50 nM, from about 50 nM to about 100 nM, from about 100 nM to about 200 nM, from about 200 nM to about 300 nM, or from about 300 nM to about 400 nM, as determined using the Hoffmann method. See Hoffman et al. (2004) Naunyn-Schmiedeberg's Arch Pharmacol 369:151-159. In various further aspects, the beta blocker can have a Kᵢ at beta receptors of less than 1 nM as determined using the Hoffmann method. As would be appreciated by one of skill, the Kᵢ of the beta blocker present can be dependent on the beta blocker being used.

In various aspects, the beta blocker can have a high Kᵢ at alpha receptors such as, for example, a Kᵢ of at least about 5,000, at least about 6,000 nM, at least about 7,000 nM, at least about 8,000 nM, or at least about 9,000 nM, as determined using the Hoffmann method. See Hoffman et al. (2004) Naunyn-Schmiedeberg's Arch Pharmacol 369:151-159. As would be appreciated by one of skill, the Kᵢ of the beta blocker present can be dependent on the beta blocker being used.

In various aspects, the beta blocker exhibits a ratio of beta:alpha blockade of at least about 10:1, at least about 100:1, at least about 500:1, at least about 1,000:1, at least about 2,000:1, or at least about 3,000:1.

In various aspects, the method further comprises using a software application to monitor one or more characteristics of the patient. In some aspects, the characteristic is one or more vital signs selected from the group consisting of body temperature, blood pressure, heart rate, respiratory rate, or any combination thereof. In some aspects, the characteristic is detected blood level of the beta blocker, and this level is used to determine whether a dosage of the MAOI, the beta blocker, or both the MAOI and the beta blocker should be adjusted during the therapeutic regimen. In some aspects, the characteristic is an indicator of patient compliance.

In various aspects, the dosage form consists of the MAOI and the beta blocker.

In various aspects, the dosage form does not contain an alpha blocker. In a further aspect, the dosage form does not contain a 5HT1A agonist.

In various aspects, one or both of the MAOI and the beta blocker is formulated as a modified-release dosage form. In a further aspect, the MAOI is formulated as a modified-release dosage form. In a still further aspect, the beta blocker is formulated as a modified-release dosage form.

In various aspects, the MAOI is phenelzine. In a further aspect, the MAOI is TCP.

In various aspects, the beta blocker is pindolol. In a further aspect, the beta blocker is carvedilol.

In various aspects, the subject is not on a tyramine restricted diet subsequent to the administering step.

### D. EXAMPLES

The present invention is further defined in the following Examples. It should be understood that these Examples, while indicating preferred aspects of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

### 1. Proposed Mechanism of Action

Herein, a MAOI-based composition designed to allow safer use for the treatment of patients with psychiatric disorders such as depression is described. Without wishing to be bound by theory, the approach detailed herein uses a mechanism-based strategy to reduce the tyramine pressor impact. Specifically, the approach is to reduce the tyramine-induced pressor response through direct blockade of norepinephrine stimulation of beta-1 adrenergic receptors and thus, to prevent an increase in cardiac output and minimize changes to heart rate and blood pressure.

Phenelzine (PHZ) is a potent, non-selective MAOI used for the treatment of depression in adults (Sidhu, G., & Marwaha, R. (2023). Phenelzine. In StatPearls. StatPearls Publishing). Phenelzine irreversibly binds to both MAO-A and MAO-B. Phenelzine inhibits the oxidation of monoamines by MAO enzymes, which is believed to result in accumulation of the endogenous catecholamines serotonin, norepinephrine, and dopamine, resulting in prolongation of their effects on their target receptors. It is unknown, however, whether MAO inhibition itself or other pharmacologic actions (or both) are responsible for the anti-depressive effects observed (Parke Davis 2007).

Tyramine is a naturally occurring monoamine compound that is endogenous to human and lower animal species in trace amounts and may be found in certain foods such as cheese or other fermented foods and drinks, often as the result of fermentation or decay-induced decarboxylation of tyrosine (Philips, S. R., et al., (1978) Biological psychiatry, 13 (1), 51-57). Tyramine acts as an indirect sympathomimetic agent that enters neurons through reuptake transporters, primarily the norepinephrine transporter, resulting in displacement of catecholamines, such as norepinephrine, from neuronal catecholamine storage sites (Da Prada et al. (1988) Pharmacol Res Commun. 20 Suppl 4, pp. 21-33 and Meck et al. (2003) J Cardiovasc Pharmacol 41 (1), pp. 126-131). The release of excess norepinephrine can result in cardiovascular effects, primarily a dose dependent increase in systolic blood pressure that, in severe cases, can lead to a dangerous hypertensive crisis.

Tyramine is metabolized by several enzymes, including MAO, catechol-O-methyltransferase, aldehyde dehydrogenase, and cytochrome P450; however, MAO-A is proposed to be the principal metabolic enzyme of tyramine. Under typical circumstances, it is estimated that less than 1% dietary tyramine ends up in systemic circulation (Schwenk (1989) Strat. Mgmt. J. 10 (3), pp. 303-306). However, when MAO is substantially inhibited by drugs such as MAOIs, significantly more tyramine is systemically available, mainly due to the reduced metabolism by MAO. With a significant increase in systemic tyramine due to blocked metabolism by an MAOI, lower oral doses of tyramine are required to elevate blood pressure, increasing the risk of a hypertensive event.

Without wishing to be bound by theory, it is believed that the downstream effect of PHZ (and other MAOIs) on tyramine accumulation and the resultant cardiovascular effects can be attenuated by the addition of a beta blocker such as pindolol (PIN).

Beta blockers are a class of drugs that block beta-adrenergic signaling and are used to treat cardiovascular conditions including hypertension (Farzam and January 2021). There are 3 types of beta receptors: beta-1 (B1), beta-2 (B2), and beta-3 (B3). B1 receptors are primarily located in the heart and mediate cardiac activity. Beta-2 receptors are located across many organ systems (especially the lungs) and mediate smooth muscle relaxation. Activation of B3 receptors is noted to induce the breakdown of fat cells. Beta blockers vary in specificity toward different receptors and the effects produced depend on the type of receptor blocked and organ system(s) involved (Farzam and January 2021).

Pindolol is a non-selective beta blocker, and it is expected that activity of pindolol on the B1 receptors in the heart will decrease heart rate and contractility leading to a decrease in blood pressure (Blumenfeld et al. 1999). In addition, pindolol may also suppress the renin-angiotensin-aldosterone system, thereby further reducing blood pressure (Blumenfeld et al. 1999). Importantly, pindolol is a noted partial agonist of beta receptors and therefore possesses intrinsic sympathomimetic activity (ISA) that is manifested as a smaller reduction in resting heart rate and cardiac output compared to beta blockers that lack ISA (Novartis 2007). The ISA of pindolol may help minimize the tiredness or “sluggishness” in patients who are administered a beta blocker, which may be important for a patient with major depressive disorder or major depressive disorder with anxious distress.

### 2. In Vitro Interference Testing

To evaluate whether inhibition of MAO-A and MAO-B by phenelzine is effected by the presence of a beta blocker, MAO inhibition enzyme assays were performed. Methods were adapted from those previously described. See, e.g., Urban et al. (1991) FEBS Lett. 286 (1-2): 142-146 and Youdim and Finberg (1991) Biochem. Pharmacol. 41 (2): 155-162. It is noted that the testing concentrations correspond to the concentrations of phenelzine sulfate and bisoprolol fumarate, the salt forms of phenelzine and bisoprolol, respectively. The free base concentration of phenelzine is equivalent to the salt form, while the free base concentration of bisoprolol is twice as high as its salt form.

Assays were performed as detailed below.

TABLE 1
Monoamine Oxidase MAO-A

| | | | |
|---|---|---|---|
| Source | Human recombinant | Pre-Incub. | 15 min. @ 37 °C |
| | Insect cells | Time/Temp. | |
| Substrate | 50.0 μM Kynuramine | Incubation | 60 min. @ 37 °C |
| | | Time/Temp. | |
| Vehicle | 1.0% DMSO | Incubation Buffer | 100 mM Potassium |
| | | | Phosphate, pH 7.4 |
| Significance Crit. | ≥50% of max | Quantitation | Spectrofluorimetric |
| | stimulation or | Method | quantitation of 4- |
| | inhibition | | hydroxquinoline |

Monoamine Oxidase MAO-B

| | | | |
|---|---|---|---|
| Source | Human recombinant | Pre-Incub. | 15 min. @ 37 °C |
| | Insect cells | Time/Temp. | |
| Substrate | 50.0 μM Kynuramine | Incubation | 60 min. @ 37 °C |
| | | Time/Temp. | |
| Vehicle | 1.0% DMSO | Incubation Buffer | 100 mM Potassium |
| | | | Phosphate, pH 7.4 |
| Significance Crit. | ≥50% of max | Quantitation | Spectrofluorimetric |
| | stimulation or | Method | quantitation of 4- |
| | inhibition | | hydroxquinoline |

Reference standards were run as an integral part of each assay to ensure the validity of the results obtained. See Table 2.

TABLE 2

| | Reference | Historical | Concurrent |
|---|---|---|---|
| Assay Name | Compound | IC₅₀ | IC₅₀ |
| Monoamine | Clorgyline | 0.93 nM | 0.77 nM |
| Oxidase | (Batch 509457) | | |
| MAO-A | Clorgyline | 0.93 nM | 0.77 nM |
| | (Batch 510277) | | |
| | Clorgyline | 0.93 nM | 0.77 nM |
| | (Batch 511430) | | |
| Monoamine | R(−)-Deprenyl | 9.80 nM | 5.63 nM |
| Oxidase | (Batch 509458) | | |
| MAO-A | R(−)-Deprenyl | 9.80 nM | 4.34 nM |
| | (Batch 510276) | | |
| | R(−)-Deprenyl | 9.80 nM | 4.54 nM |
| | (Batch 511432) | | |

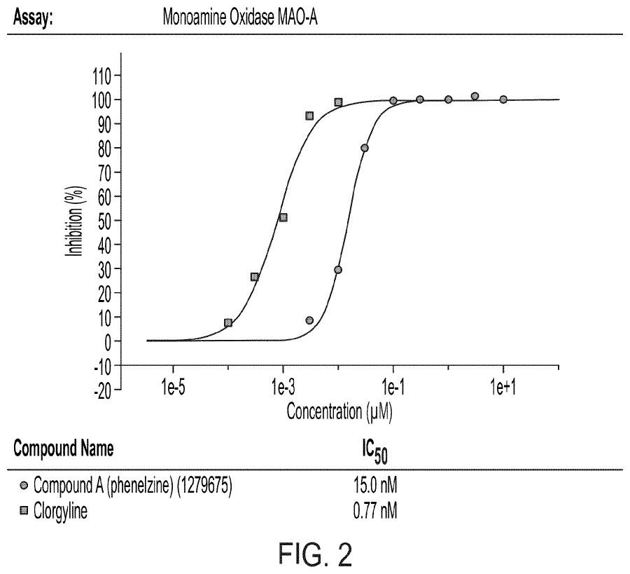

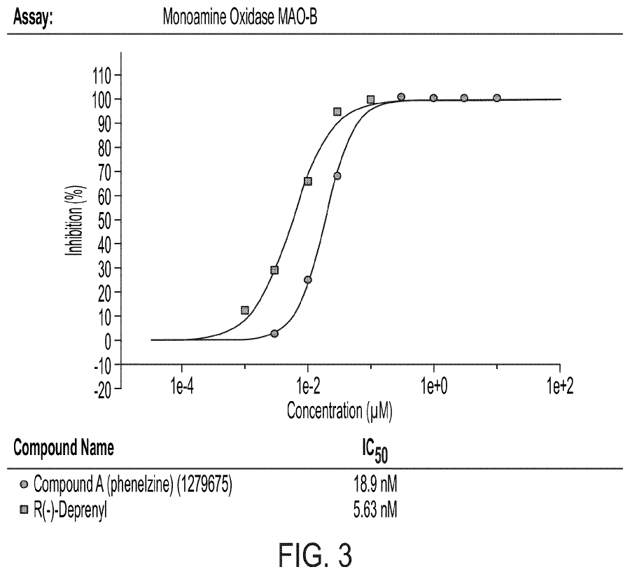

First, the IC₅₀ of phenelzine with MAO-A and MAO-B was determined. See Table 3, FIG. 2, and FIG. 3. The IC₅₀ of phenelzine with MAO-A and MAO-B was determined to be 15 nM and 18.9 nM, respectively, from the fitted curve.

TABLE 3

| Phenelzine | % MAO-A | % MAO-B |
|---|---|---|
| Concentration (nM) | inhibition | inhibition |
| 300 | 100 | 101 |
| 100 | 100 | 100 |
| 30 | 80 | 68 |
| 10 | 30 | 25 |
| 3 | 8 | 3 |

Next, binding of the beta blockers pindolol, bisoprolol, and carvedilol to MAO-A and MAO-B was evaluated at concentrations relevant to typical dosing of these drugs. As shown in Table 4 below, no significant inhibitory activity of MAO-A and MAO-B was observed. Criteria for significant responses in the biochemical assays was defined as ≥50% inhibition.

TABLE 4

| | % MAO-A | % MAO-B |
|---|---|---|
| Beta Blocker | inhibition | inhibition |
| Pindolol (2 μM) | −1 | −5 |
| Bisoprolol (2 μM) | −5 | 13 |
| Carvedilol (2 μM) | 4 | 23 |

The interference of MAO-A and MAO-B inhibition by phenelzine in the presence of either pindolol, bisoprolol, or carvedilol was then evaluated, using both low (IC₅₀ was determined to be 15 nM with MAO-A and 18.9 nM with MAO-B; Tables 5 and 6) and high (150 nM; Table 7) doses. As shown, no interference was observed for the inhibition of MAO-A and MAO-B with phenelzine in the presence of the individual beta blockers. In other words, phenelzine inhibits MAO-A and MAO-B to the same degree in the presence and absence of beta blockers.

TABLE 5

| | | MAO-A % |
|---|---|---|
| MAO inhibitor | Beta Blocker | inhibition |
| Phenelzine (15 nM) | none | 38 |
| Phenelzine (15 nM) | Pindolol (2 μM) | 39 |
| Phenelzine (15 nM) | Bisoprolol (0.44 μM) | 34 |
| Phenelzine (15 nM) | Carvediol (0.52 μM) | 35 |

TABLE 6

| | | % MAO-B |
|---|---|---|
| MAO inhibitor | Beta Blocker | inhibition |
| Phenelzine (18.9 nM) | none | 55 |
| Phenelzine (18.9 nM) | Pindolol (2 μM) | 54 |
| Phenelzine (18.9 nM) | Bisoprolol (0.44 μM) | 55 |
| Phenelzine (18.9 nM) | Carvediol (0.52 μM) | 51 |

TABLE 7

| | | % MAO-A | % MAO-B |
|---|---|---|---|
| MAO inhibitor | Beta Blocker | inhibition | inhibition |
| Phenelzine (150 nM) | Pindolol | 101 | 100 |
| | (2 μM) | | |
| Phenelzine (150 nM) | Bisoprolol | 100 | 100 |
| | (0.44 μM) | | |
| Phenelzine (150 nM) | Carvediol | 93 | 99 |
| | (0.52 μM) | | |

### 3. Inhibition of MAO-A and MAO-B by Isocarboxazid

To evaluate the inhibition of MAO-A and MAO-B by isocarboxazid at 10 μM MAO inhibition enzyme assays were performed as described above and in Table 1.

Reference standards were run as an integral part of each assay to ensure the validity of the results obtained. See Table 8.

TABLE 8

| | Reference | Historical | Concurrent |
|---|---|---|---|
| Assay Name | Compound | IC₅₀ | IC₅₀ |
| Monoamine Oxidase | Clorgyline | 0.93 nM | 0.95 nM |
| MAO-A | (Batch 510404) | | |
| Monoamine Oxidase | R(−)-Deprenyl | 9.80 nM | 6.04 nM |
| MAO-A | (Batch 510459) | | |

The inhibition of isocarboxazid at 10 μM with MAO-A and MAO-B was determined. See Table 9.

TABLE 9

| Isocarboxazide | % MAO-A | % MAO-B |
|---|---|---|
| Concentration (μM) | inhibition | inhibition |
| 10 | 91 | 98 |

### 4. In Vivo Rat Telemetry Study #1

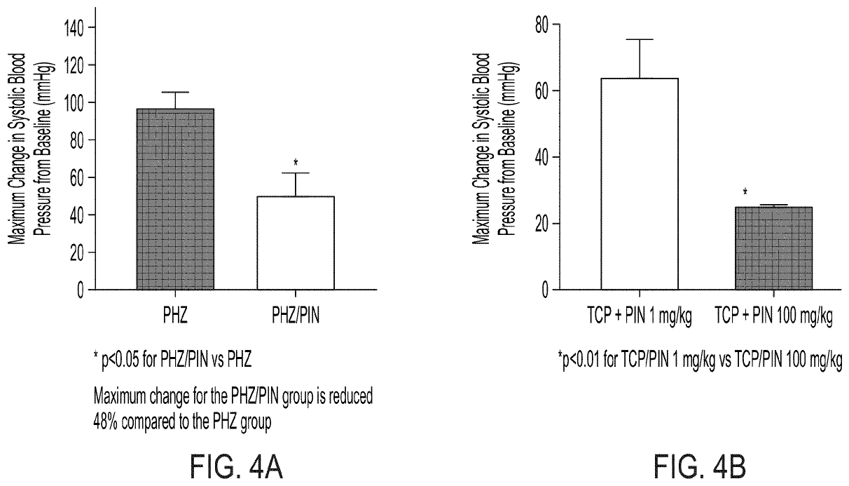

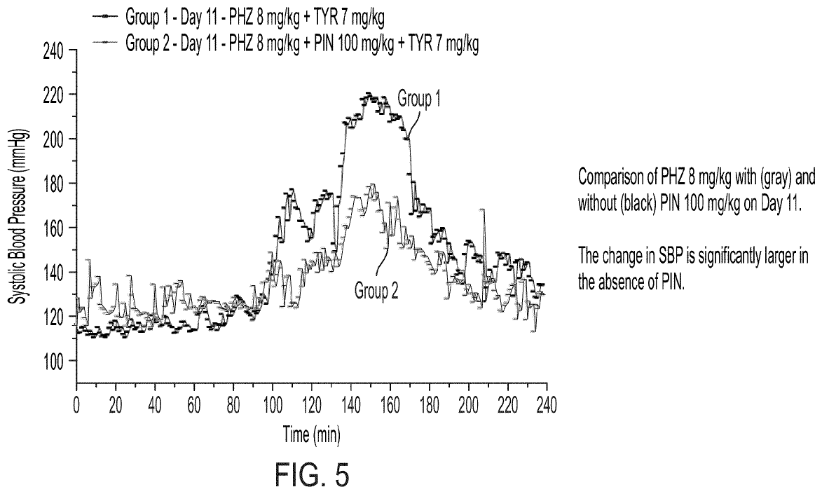

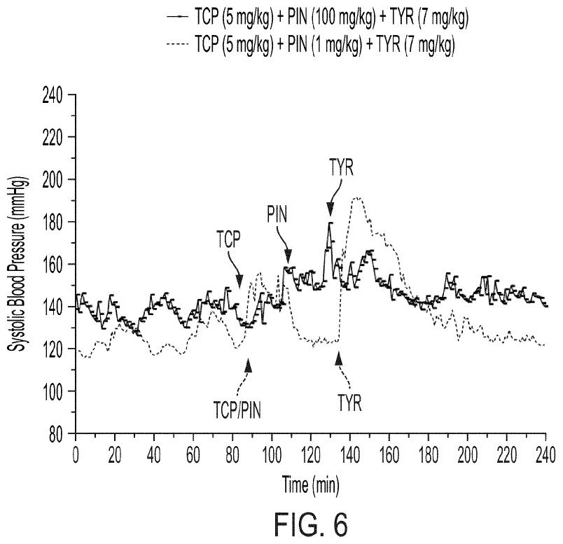

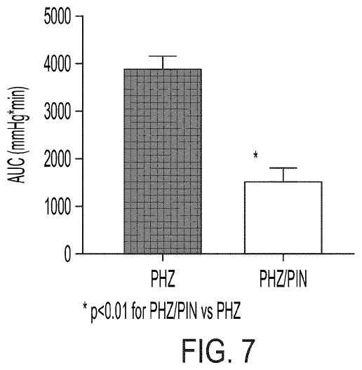

To evaluate the effect of the combination of a MAOI and a beta blocker on changes to blood pressure after a tyramine dose, an in vivo study in rats implanted with telemetry transmitters was completed, and the rats were allowed to recover for 7 days. Briefly, phenelzine (50 mg/kg for the first dose and then daily 8 mg/kg) was administered orally alone (N=3) or in combination with pindolol (100 mg/kg) (N=4). Pindolol is a non-selective beta-blocker with ISA. The maximum change in systolic blood pressure following an oral tyramine dose (7 mg/kg) between 10-60 minutes after MAOI and beta-blocker administration is shown in FIG. 4A. A similar combination study with tranylcypromine (8 mg/kg for the first dose and then daily 5 mg/kg) as the MAOI administered in combination with pindolol (1 mg/kg [N=3] or 100 mg/kg [N=3]) was also completed (FIG. 4B). For phenelzine, the maximum change in systolic blood pressure following oral tyramine is reduced by 48% if pindolol is administered sequentially with the MAOI. For tranylcypromine, the maximum change in systolic blood pressure following oral tyramine administration is reduced in a dose dependent manner when different pindolol dose levels are administered. A time course of systolic blood pressure in the rat for phenelzine (8 mg/kg) alone or in combination with pindolol (100 mg/kg) following administration of tyramine (7 mg/kg) is shown in FIG. 5. A similar time course with tranylcypromine (5 mg/kg) with pindolol at two doses (1 mg/kg and 100 mg/kg) following administration of tyramine (7 mg/kg) is shown in FIG. 6. The area under the curve (AUC) for systolic blood pressure is also reduced for phenelzine with pindolol by 61% compared to phenelzine alone (FIG. 7). The reduction in maximum change in systolic blood pressure and AUC, as well as the time courses demonstrate that the change in systolic blood pressure from administration of tyramine during MAOI treatment is reduced when pindolol is administered sequentially.

### 5. In Vivo Rat Telemetry Study #2

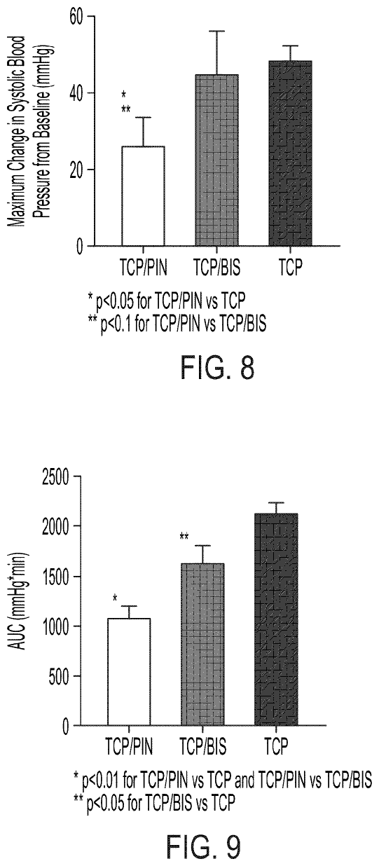

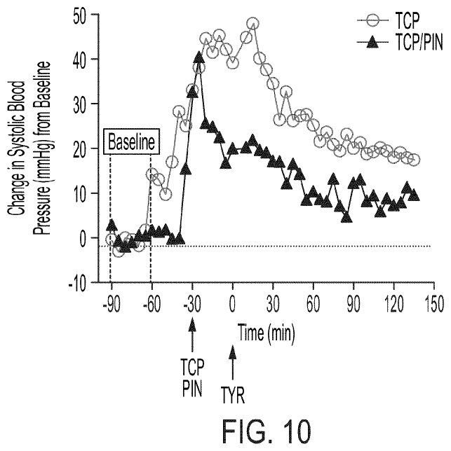

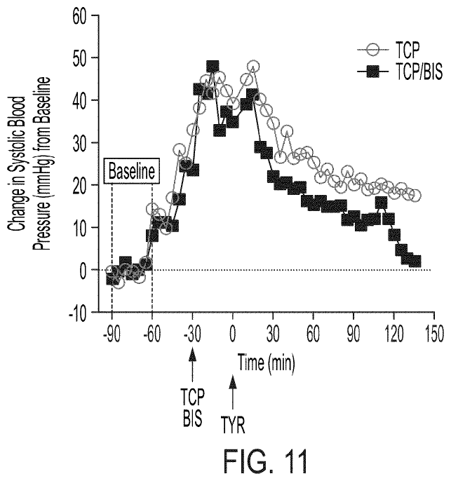

To evaluate the effect of the combination of a MAOI and a beta blocker on changes to blood pressure after a tyramine dose, an in vivo study in rats implanted with telemetry transmitters was completed, and the rats were allowed to recover for 7 days. Briefly, rats received tranylcypromine (8 mg/kg for the first dose and then daily 5 mg/kg) alone (N=5), with pindolol (60 mg/kg, N=5) or with bisoprolol (20 mg/kg, N=5). Pindolol is a non-selective beta-blocker with ISA. Bisoprolol is a selective beta-blocker without ISA. On Day 8, in addition to the described daily dosing, tyramine (3 mg/kg) was administered 30 minutes after tranylcypromine and pindolol or bisoprolol administration, and blood pressure was monitored. The maximum change in systolic blood pressure was 46% and 7% lower for tranylcypromine with pindolol or bisoprolol, respectively, compared to tranylcypromine alone (FIG. 8). The AUC for systolic blood pressure is also reduced for tranylcypromine with pindolol or bisoprolol by 49% and 23%, respectively, compared to tranylcypromine alone (FIG. 9). A time course with baseline systolic blood pressure subtracted of tranylcypromine (5 mg/kg) compared to tranylcypromine with pindolol (60 mg/kg) or bisoprolol (20 mg/kg) following administration of tyramine (3 mg/kg) is shown in FIG. 10 and FIG. 11, respectively. The reduction in maximum change and AUC of the systolic blood pressure and the differences in time course for each group demonstrate that the change in systolic blood pressure from administration of tyramine during MAOI treatment is reduced when pindolol is administered sequentially. Additionally, there is a reduction in change in systolic blood pressure from administration of tyramine during MAOI treatment when bisoprolol is administered sequentially, but the magnitude of reduction is less than pindolol.

### 6. In Vivo Rat Telemetry Study #3

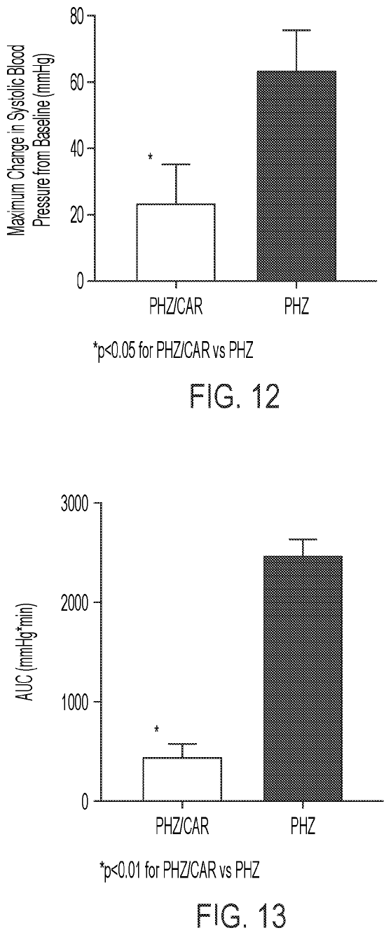

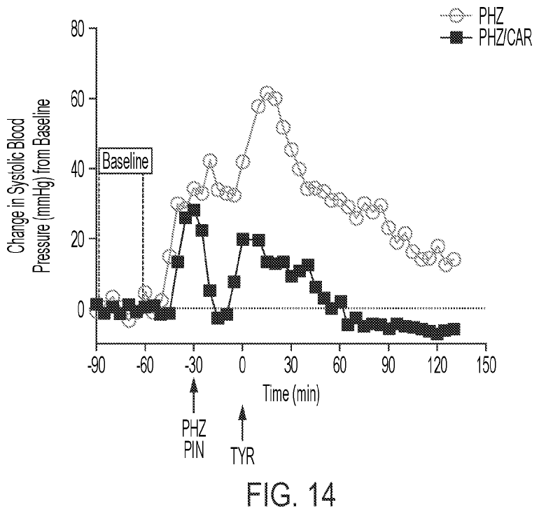

In another study to evaluate the effect of the combination of a MAOI and a beta blocker on changes to blood pressure after a tyramine dose, an in vivo study in rats implanted with telemetry transmitters was completed, and the rats were allowed to recover for 7 days. After recovery, rats received phenelzine (8 mg/kg daily) alone (N=5) for 3 days and with carvedilol (50 mg/kg, N=5) for 4 days. Carvedilol is a non-selective beta-blocker without ISA. On Day 8, in addition to the described daily dosing of phenelzine and carvedilol, tyramine (3 mg/kg) was administered 30 minutes after phenelzine and carvedilol administration, and blood pressure was monitored. The maximum change in systolic blood pressure was 63% lower for phenelzine with carvedilol compared to phenelzine alone (FIG. 12). The AUC for systolic blood pressure is also reduced for phenelzine with carvedilol by 82% compared to phenelzine alone (FIG. 13). A time course with baseline systolic blood pressure subtracted of phenelzine (8 mg/kg) compared to phenelzine with carvedilol (50 mg/kg) following administration of tyramine (3 mg/kg) is shown in FIG. 14. The reduction in maximum change and AUC of the systolic blood pressure and the time course demonstrate that the change in systolic blood pressure from administration of tyramine during MAOI treatment is reduced when carvedilol is administered sequentially.

### 7. Evaluation of the Effect of the Combination of a MAOI and a Beta Blocker on Transient Changes to Systolic Blood Pressure

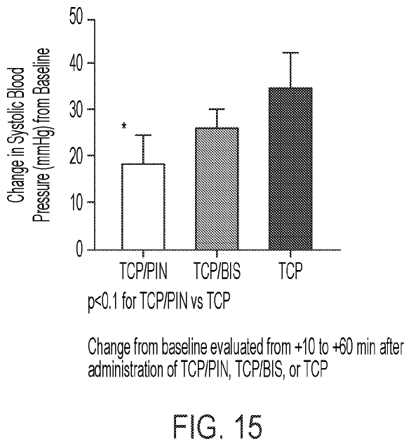

Significant transient blood pressure increases (20 mmHg or more in either systolic or diastolic blood pressures) may occur in approximately 50% of patients immediately after they take either MAOIs phenelzine or tranylcypromine, which return to normal after 1 to 2 hours (Keck et al. (1989) J. Clin. Psychopharmacol. 9(3): 203-6; Lott R. AAPP Pharmacist Toolkit: Monoamine Oxidase Inhibitors [Internet]. Lincoln, NE: American Association of Psychiatric Pharmacists, 2022). To evaluate the effect of the combination of a MAOI and a beta blocker on transient changes to systolic blood pressure, an in vivo study in rats implanted with telemetry transmitters was completed, and the rats were allowed to recover for 7 days. Briefly, rats received tranylcypromine (8 mg/kg for the first dose and then daily 5 mg/kg) alone (N=4), with pindolol (60 mg/kg, N=5) or with bisoprolol (20 mg/kg, N=5) for five days. Pindolol is a non-selective beta-blocker with ISA. Bisoprolol is a selective beta-blocker without ISA. On Day 5, a comparison of the change from baseline of systolic blood pressure was evaluated for each group from +10 to +60 minutes after administration of MAOI alone or MAOI with beta-blocker. Compared to tranylcypromine alone, the tranylcypromine and pindolol group had a 46% reduction in systolic blood pressure increase from baseline following administration, and the tranylcypromine and bisoprolol group had a 24% reduction (FIG. 15). Without wishing to be bound by theory, these results demonstrate that the combination of tranylcypromine with pindolol or bisoprolol reduces transient blood pressure increases following administration compared to tranylcypromine alone.

### 8. Prophetic Manufacturing Protocol for the Dosage Form

The combination of phenelzine sulfate and pindolol [1-(Indol-4-yloxy)-3-(isopropylamino)-2-propanol] will be manufactured as a single dosage form, either tablet, capsule, suppository, injection, or other delivered dose. The dosage form(s) will deliver the two compounds at targeted pharmacological levels with concomitant delivery to maintain the effective level of phenelzine and protective level of pindolol for safety of this fixed dose drug/drug combination.

Each active pharmaceutical ingredient (API) can be formulated together or separately. Each matrix (API with corresponding excipients) will be developed to account for the drug solubility and pharmacology unique to its chemistry. The API matrices will be combined into single dosage form and presented to the patient in a single dosage form or concomitantly administered as two APIs. Pharmaceutical grade excipients can be incorporated in the formulation(s) achieve the desired release profile individually. The modified-release profile can consist of micro particulates, pellets, or granulation coated or uncoated in tablet, capsule, suppository, or suspension. As detailed elsewhere herein, a modified-release dosage form refers to any dosage form in which the drug substance solubility or availability in the dosage form is modified or altered. This includes terms such as sustained, extended, delayed, enteric, colonic, pulsatile, and bimodal. Tablets can be compressed as single, bi-layer, or multi-layer, and can be coated or uncoated. Tablet coating can be functional for the release profile. Drug release can be achieved through other methods such as reservoir polymeric systems or osmotic pump systems with laser drilled tablets or capsules.

Typical binding agents for solid oral dosage forms (tablets and capsules) include the following: pregelatinized maze starch or hydroxypropyl methylcellulose of varying degrees of substitutions; fillers (e.g., lactose, microcrystalline cellulose, calcium carbonate or calcium phosphate); disintegrants (potato starch, croscarmellose Sodium, or Sodium starch glycolate); wetting agents (e.g., Sodium lauryl Sulphate or non-ionic surfactants) or other agents suitable for tableting.

Inactive ingredients for tablet, pellet, or spheres for matrix release include the typical ingredients previously identified and nonionic homopolymers of ethylene oxide, water soluble natural gums of polysaccharides water swellable but insoluble, high molecular weight homopolymers and copolymers of acrylic acid chemically crosslinked with polyalkenyl alcohols, polyvinyl acetate and povidones, crosslinked amylose starch and ionic methacrylate copolymers. Additionally fatty acids, fatty acid esters and mono-, di-, tri-glycerides of differing melting points as well as hydrophobic polymers, natural occurring waxes, and ammoniomethacrylate copolymers can be used in non-swellable matrix. Lipid or hydrophobic matrices can delay the release of the drug and can also be used in the delayed-release technology.

Coating materials for modified-release of drug(s) from formulated matrices (tablets, pellets, or spheres), which includes enteric and other release targets include shellac and zein of natural sources, derivatives of cellulose (cellulose acetate phthalate, hydroxypropyl methylcellulose phthalate) and methacrylic (acid and ester copolymers containing carboxylic functional groups) that resist solubility low pH and protect the delivery to other pH greater than 5.5. Plasticizers (such as triacetin, triethanol citrate, glycerin, and polyethylene glycol) may or may not be needed to facilitate the release profile and flexibility of the coating.

Materials for reservoir systems use the ingredients previously mentioned for drug release coatings surrounding the drug core matrix (most commonly a tablet) to form a controlled barrier, which can be a water insoluble coating such as cellulose acetate. The core of the tablet can contain an osmotic agent such as sodium chloride or other hydrophilic polymers to push the drug out of the laser drilled orifice as designed.

Additional exemplary methods of manufacturing include the following: drug matrix preparation includes the intimate mixture of the drug and excipients to influence the drug behavior in the manufacture and drug delivery in the dosage form; granulation, compacting, hot melt and cold melt pelletizing and spherization. The resulting matrix (granulation, blend, pellet or sphere) can be a ready dosage form with tableting or encapsulation or further manipulated for the desired release profile.

Granulation of the drug matrix can be compressed/tableted as a single layer or multiple layer tablet. This tablet can be coated with coating for further control of the drug release from the tablet core. Coating can be functional for modified-release of the core or contain additional drug product for release in a multi-modal profile.

Granulation or drug matrix can be further manipulated to form small pellets or spheres with methodology such as extrusion spheronization and rotary processing. Extrusion and pelletizing drug matrices are known methodologies used for lipid nanoparticles and pellets.

Spraying of drug onto inert or drug matrix spheres followed by coating in equipment designed to handle small beads/pellets/spheres. This drug loaded sphere can be further coated to deliver a controlled/modified release.

Tableting methodology has many options of compressing a core tablet within an outer tablet or multiple layer tablets. The compression of the granulation or powder drug matrix can be achieved through controlled pressure and appropriate dyes for the tablet formation. Pellets, granulation, or prepared spheres can be delivered to capsule shells, which are designed to be immediate or modified release.

### 9. Prophetic Pharmacokinetic Studies

a. Dissolution

Modified-release formulations will initially be tested in vitro by dissolution and compared to dissolution results from the immediate release. Dissolution methods are well-known by those of skill in the art. Without wishing to be bound by theory, it is believed that the release of both phenelzine and pindolol from the modified-release formulation will be significantly slower than the release of the same drug from the immediate release drug product.

b. In Vivo Rat Studies

Single dose PK studies in rats will include doses of phenelzine ranging from about 2 to 20 mg/kg and of pindolol ranging from about 2 to 8 mg/kg. The doses will be selected based on conversion of the typical human dose of phenelzine and pindolol to rat based on body surface area (60 kg human; 0.3 kg rat; scaling factor of 6.2) and will take into account potential differences in metabolism between species where understood. Doses will be administered by oral gavage of modified-release formulations of phenelzine and pindolol. Blood samples will be collected at approximately pre-dose, 0.25, 0.5, 1, 2, 4, 8, 16, and 24 hours. Plasma will be isolated from blood and frozen until analysis by LC/MS/MS to measure plasma phenelzine and pindolol concentrations compared to immediate release formulations. The phenelzine and pindolol plasma concentration data will be used to calculate pharmacokinetic parameters including Cₘₐₓ, Tₘₐₓ, AUC₀₋₂₄, AUC_{0-inf}, V/F, Cl/F, kₐ and kₑ.

c. In Vivo Dog Studies

Single dose PK studies in dogs will include doses of phenelzine ranging from about 1 to 4 mg/kg and of pindolol ranging from about 0.5 to 4 mg/kg. The doses will be selected based on conversion of the typical human dose of phenelzine and pindolol to dog based on body surface area (60 kg human; 8 kg dog; scaling factor of 1.8) and will take into account potential differences in metabolism between species where understood. Doses will be administered orally by capsule or tablet containing modified-release formulations of phenelzine and pindolol. Blood samples will be collected at approximately pre-dose, 0.25, 0.5, 1, 2, 4, 8, 16, and 24 hours. Plasma will be isolated from blood and frozen until analysis by LC/MS/MS to measure plasma phenelzine and pindolol concentrations compared to immediate release formulations. The phenelzine and pindolol plasma concentration data will be used to calculate pharmacokinetic parameters including Cₘₐₓ, Tₘₐₓ, AUC₀₋₂₄, AUC_{0-inf}, V/F, Cl/F, kₐ and kₑ.

d. Predicted Pharmacokinetics of the Modified-Release Formulations

Without wishing to be bound by theory, the pharmacokinetics of the modified-release formulations are expected to result in the following changes for phenelzine: (1) significant decrease in Cₘₐₓ compared to the immediate release formulation, e.g., up to 60%; (2) significant delay in Tₘₐₓ compared to the immediate release formulation, e.g., up to 10 hrs (similarly, this is reflected in a decrease in kₐ); (3) decrease or similar AUCs compared between the modified and immediate release formulations; and (4) phenelzine pharmacokinetics will support a once-daily dosing regimen of the modified-release formulation compared to bid for the immediate release tablet.

Without wishing to be bound by theory, the pharmacokinetics of the modified-release formulations are expected to result in the following changes for pindolol: (1) significant decrease in Cₘₐₓ compared to the immediate release formulation, e.g., up to 80%; (2) significant delay in Tₘₐₓ compared to the immediate release formulation, e.g., up to 10 hrs (similarly, this is reflected in a decrease in kₐ); (3) decrease or similar AUCs compared between the modified and immediate release formulations; (4) plasma concentration time curve that demonstrates a minimum plasma concentration of 6 ng/mL pindolol over a 24 hour period (this correlates with a prediction of 85% beta blockade based on the pindolol Kᵢ and plasma levels, and may be important for blocking the tyramine effect); and (5) pindolol pharmacokinetics will support a once-daily dosing regimen of the modified-release formulation compared to bid for the immediate release tablet.

### 10. Prophetic Nonclinical Safety Study in Beagle Dogs

The nonclinical safety study provided herein will be conducted using beagle dogs. In these studies, the test subjects will be given an oral dosage form once daily that includes a control group receiving vehicle only, a modified-release dosage form of phenelzine and pindolol in combination at either a high or a low dose, a high dose of phenelzine, or a high dose of pindolol. The test subjects will be compared.

Endpoints include: mortality/cageside observations, detailed clinical observations, body weight, food consumption, ophthalmology, electrocardiogramaspiratory assessment, clinical pathology (hematology, clinical chemistry, coagulation, urinalysis), organ weights, histopathology (full set of tissues), and toxicokinetic analysis for pindolol and phenelzine and their major metabolites (after first administration and prior to end of study).

### 11. Prophetic Phase 1 Study: Combination Dose-Finding PK and Tyramine Challenge Study

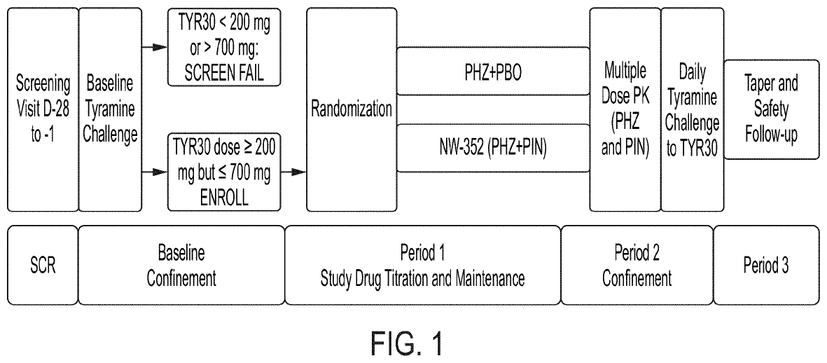

A placebo-controlled, parallel group design to evaluate the potential of NW-352 (PHZ+PIN) to minimize the BP elevation following oral administration of fasted tyramine (TYR) will be conducted (FIG. 1). An analysis will be performed to compare the treatment effects, including determination of TYR30 and calculation of TSF values, between NW-352 and phenelzine plus placebo (PHZ+PBO) treatment groups when 6 participants have completed their treatment in each group.

Eligibility will be assessed during the Screening Period. Participants meeting entry criteria will start the Baseline Period to determine enrollment with a pre-enrollment TYR challenge series in the clinical research unit (CRU). One TYR challenge will be conducted per day until TYR30 is achieved. Those whose eligibility is confirmed (TYR30≥200 mg and ≤700 mg) will enter Period 1 (open-label PHZ and blinded PIN/PBO)

A TYR dosing advancement procedure (also referred to as TYR algorithm), which is based on the approximate linear relationship between TYR and changes in SBP, will be applied to determine if any single dose of TYR in the sequence can be skipped during the pre-treatment and on-treatment TYR challenges.

a. Pre-Treatment Baseline TYR Challenge

The pre-treatment (pre-enrollment) fasting TYR challenges are used as a final screening procedure to determine enrollment. The sequence of TYR doses is provided in the section below TYR Challenges-Algorithm Description. TYR challenges will continue until TYR30 is achieved or the end of the TYR dosing sequence is reached.

After the Baseline TYR challenges are complete and Baseline TYR30 is determined, PHZ will be titrated to a maintenance dose of 30 mg BID with either 5 mg BID PIN or PBO. Day 1 of Period 1 commences in the afternoon in the CRU following the last TYR challenge of the Baseline Period. Participants will be randomized (1:1) to one of two study drug treatments dosed approximately 12 hours apart, as follows: (a) PHZ 30 mg BID+PBO BID; and (b) NW-352 (PHZ 30 mg BID+PIN 5 mg BID). PHZ is given open-label, while the PIN and PBO capsules are blinded.

After approximately 27 days of study drug dosing in Period 1, participants will be admitted to the CRU on Period 1, Day 27. Period 2 begins the day after admission with a 12-hour PK sampling, after the study drug morning dosing. The On-treatment TYR challenge series will be administered to allow treatment comparisons in response to TYR when treated with either NW-352 or PHZ+PBO.

b. On-Treatment TYR Dosing

For Period 2, when participants are on NW-352 and PHZ+PBO, TYR dosing will be determined using the sequences described below in TYR Challenges—Algorithm Description. In comparison to the pre-treatment TYR challenge, it is expected that lower doses of TYR would be required to reach a TYR30 for the participants on PHZ+PBO. TYR challenges will continue until TYR30 is achieved or the end of the TYR dosing sequence is reached.

Participants will remain confined in the CRU during all challenges.

In Period 3, participants will be discharged to complete a taper of the study drugs over approximately 6 days, with a Safety Follow-up Visit on approximately Day 7 of Period 3, and a Safety Phone call on approximately Day 18.

a. TYR Challenges—Algorithm Description

In general, only 1 TYR challenge will be given per day. With noted exceptions, all TYR challenges will begin 2 hours after the AM dose of study drug is given.

The objective of the TYR dosing sequence or algorithm is to gradually advance the dose of TYR until TYR30 is achieved. For each participant, the TYR challenge begins with the lowest TYR dose in the sequence associated with the participant's treatment arm. The dosing procedure provided is designed to advance safely through the TYR doses to TYR30, while minimizing risk and burden to the participants. The procedure is based on published literature that demonstrates there is an approximately linear relationship between TYR and changes in SBP (Cantarini et al. 2004, Schafers et al. 1999, Freychuss et al. 1970). This procedure may be applied to determine if any single dose of TYR in the sequence can be skipped.

Unblinded pharmacists and physician(s) will manage the TYR dosing advancement procedure and blinded TYR dosing utilizing the safety and SBP data from each TYR challenge that is available for each participant.

Dosing Sequences: For pre-randomization fasting TYR challenges: TYR doses will be 100, 200, 300, 400, 500, 600, and 700 mg. For on-treatment fasting TYR challenges for PHZ+PBO, which will be selected based on the algorithm: TYR doses will be 5, 10, 25, 35, 50, 75, 100, 125, 150, 200 mg. For on-treatment fasting TYR challenges for PHZ+PIN, which will be selected based on the algorithm: TYR doses will be 10, 25, 50, 100, 150, 200, 300, 400, 500, 600, 700 mg.

Oral TYR challenges are designed to elicit a subject's increase in SBP relative to TYR doses. TYR30 is the dose of TYR required to raise the SBP by ≥30 mmHg (measured as 3 consecutive measurements taken every 5 min within 2 hours of a fasting TYR administration) as compared to a baseline average taken pre-dose of study medication. The pre-dose baseline SBP is defined as an average of 5 SBP measurements taken 5 minutes apart, during the 30 minutes immediately prior to the initiation of each TYR challenge. All measurements will be done in the semi-recumbent position and the subject will remain in the semi-recumbent position throughout the pre-dose period and for the duration of the TYR challenge monitoring period.

TYR30 will be observed for by monitoring BP at the following frequency: Q 5 minutes for 2 hours. Q 15 minutes (for 2 additional hours for fasting challenges). If at any point the subject is not considered stable by the PI, a more frequent VS schedule may be implemented.

The TYR dose for the PHZ+PBO and PHZ+PIN alone challenge will be determined using an algorithm. The change in TYR dose for the study drug PHZ+PBO and PHZ+PIN is described by the tyramine sensitive factor (TSF) (TYR30 pretreatment/TYR30 post-treatment). Following the algorithm, it is anticipated that the subjects' TYR30 should be elicited within approximately 3 to 5 days.

Blood sampling for PK of TYR, PIN, and PHZ will be done throughout the study to evaluate the concentrations of each of these at various time points.

### 12. Prophetic Phase II Study: Pivotal Tyramine Challenge Study and Evaluation of Tyramine Pressor Response in Patients Receiving Combination Therapy of MAOI and Beta Blocker

a. Tyramine Sensitivity Factor (TSF)

TYR30 is a metric defined as the smallest dose of tyramine that raised systolic blood pressure (SBP) by at least 30 mmHg, for 3 consecutive measurement time points within 2 hours after administration of tyramine. TYR30 is determined by a clinical trial, where subjects are given progressively higher doses of oral tyramine under fasting conditions, and blood pressure is measured regularly, typically every 5 minutes for 2 hours after administration of tyramine. An SBP baseline is established before tyramine administration. As the tyramine dose is absorbed, SBP would typically rise by an amount that is dependent on the dose of tyramine and varies by individual subject. The SBP Peak is typically 30-60 minutes after tyramine dose administration with a maximum of 2 hours. The experiments start with a low dose of tyramine to ensure subject safety and is iteratively increased according to a predetermined dose sequence. TYR30 is determined as the smallest TYR dose that produces 3 consecutive measurement point where the SBP increase over baseline is greater than or equal to 30 mmHg.

The TSF is designed to determine the potentiation of tyramine by the test drug, MAOI for example. It requires the determinations of two TYR30 measurement: (1) Pre-treatment TYR30, which is the TYR30 that would be achieved by tyramine without the test drug. (2) Post-treatment TYR, which is the TYR30 that would be achieved by tyramine after the test drug has been given at a therapeutic dose, and long enough for the test drug to reach steady-state.

TSF is calculated as the ratio of pre-treatment TYR30 to post treatment TYR30. A ratio greater than 1 indicates that a lower dose of tyramine can increase SBP by 30 mmHg when the drug is taken compared to when it is not. It can also be interpreted as the level of potentiation of the tyramine pressor response. Most drugs that interact with tyramine and have been approved without required dietary restrictions, have a TSF in the range of 1.5 to 3.5. By contrast, the TSF of phenelzine at 45 mg/day is 13.3 and the TSF of tranylcypromine at 20 mg/day is 55 (Bieck and Antonin (1989) J Neural Transm. Suppl 28, pp. 21-31). A substantial tyramine attenuation is (5-15×) is required to get within the TSF range of drugs approved without dietary restrictions. Achieving those types of levels depends on the chosen beta blocker and its dosage. Care must be given to adverse effects associated with higher doses. Consideration must also be given for the tyramine attenuation at the trough of beta blocker concentration where the beta blockade is lowest, and the tyramine attenuation is weakest.

A model was developed to link the dosage of a given beta blocker with an estimated TSF for some several key MAOIs and beta blockers. It involves the following steps: (1) Establish an estimated free plasma concentration table for each beta blocker between 1 and 24 hours after a single dose. (2) Estimate the beta blockade associated with the beta blocker dose, at two time points: 2 and 6 hours after the beta blocker dose is given. (3) Using a competitive binding model, estimate the percentage of beta receptors bound by norepinephrine for a tyramine dose that increases SBP by approximately 60 mmHg when given with a placebo pill. Use the model to estimate the SBP increase when a given dose of beta blocker, integrating steps 1 through 3. (4) Calculate the estimated TSF of an MAOI when given with the beta blocker dose in step 3.

Table 10 shows the estimated tyramine SBP pressor response attenuation when a dose of tyramine that produces 60 mmHg SBP pressor response is given with placebo compared to when the same dose of tyramine is given with a beta blocker. Attenuation is shown as the ratio of the SBP pressor response with a beta blocker as compared to the SBP pressor response without a beta blocker.

TABLE 10

| | SBP Pressor Response Attenuation* with a Beta Blocker | | | |
|---|---|---|---|---|
| | | | | Fold- |
| | | TYR | TYR | difference |
| | BB Morning | Attenuation | Attenuation at | in TYR |
| Beta Blocker | Dose (mg) | at Tmax | Tmax + 6 h | attenuation |
| Atenolol | 30 | 3.5 | 2.4 | 1.5 |
| Bisoprolol | 10 | 3.6 | 2.7 | 1.3 |
| Nebivolol | 10 | 1.1 | 1.1 | 1.0 |
| Propranolol | 40 | 2.4 | 1.6 | 1.5 |
| Metoprolol | 50 | 4.9 | 2.7 | 1.81 |
| Pindolol | 5 | 26.0 | 8.6 | 223.6 |

Table 11 shows the range of estimated TSFs based on the historically reported MAOI ranges without a beta blocker, and a sensitivity analysis of the model above. The calculated reduction of MAOI TSF at 6 hours after morning beta blocker dose is shown. Range is representative of literature-reported TSF values for each of the MAOIs.

TABLE 11

| | | Phenelzine | | Tranylcypromine | | Selegiline | |
|---|---|---|---|---|---|---|---|
| | BB | 60 mg/d | | 20 mg/d | | 10 mg/d | |
| | Morning | TSF | TSF | TSF | TSF | TSF | TSF |
| Beta Blocker | Dose (mg) | Low | High | Low | High | Low | High |
| Placebo | — | 9 | 30 | 30 | 55 | 3 | 4 |
| Atenolol | 30 | 4.9 | 16.9 | 16.4 | 30.9 | 1.4 | 2.0 |
| Bisoprolol | 10 | 4.5 | 15.2 | 15.1 | 27.8 | 1.3 | 1.8 |
| Nebivolol | 10 | 8.7 | 41.6 | 28.8 | 50.9 | 2.4 | 4.9 |
| Propranolol | 40 | 6.5 | 25.4 | 21.6 | 46.6 | 1.8 | 3.0 |
| Metoprolol | 50 | 4.3 | 13.9 | 14.2 | 25.5 | 1.2 | 1.6 |
| Pindolol | 5 | 1.4 | 3.7 | 4.5 | 6.7 | 0.4 | 1.0 |

b. Tyramine Challenge Study

The study herein includes a small group of volunteers who are suffering from major depressive disorder with anxious distress. The subjects are given a placebo, a disclosed modified-release dosage form of phenelzine and pindolol, or phenelzine.

The TSF for each arm will be estimated as a first step for Tyr30 dose at baseline (pre-treatment) divided by Tyr30 during treatment (post-treatment) for each subject individually. The TSF values will be summarized by treatment.

As a study validity comparison, TSF for phenelzine alone will be compared to TSF for placebo treatment arm. The parallel comparison will be done using ANOVA/ANCOVA model for log-transformed TSF to estimate GMR and 90% confidence intervals (CI). To demonstrate sufficient sensitivity of the study, the lower limit of 90% CI for TSF, GMR has to exceed 125% to demonstrate higher range of TSF of phenelzine vs placebo (superiority).

The main comparison using the same approach will be for the modified-release dosage form vs placebo. The evaluation would include the comparisons of upper limit of 90% CI for GMR of TSF of the drugs approved without labeling restrictions to modified-release dosage form from a fasted tyramine challenge to be considered clinically significant.

During the week following the tyramine challenges for TSF measurement, subjects are tested for reaction to a tyramine rich meal under 3 different standard meal types: (1) light meal with primarily carbohydrates, (2) normal lipid rich meal, and (3) normal protein rich meal. The evaluation of this data will be focused on systolic blood pressures during tyramine challenges with meals (on-treatment) of ≤30 mmHg.

It will be apparent to those skilled in the art that various modifications and variations can be made without departing from the scope or spirit of this disclosure. Other embodiments will be apparent to those skilled in the art from consideration of the specification and practice disclosed herein. It is intended that the specification and examples be considered as exemplary only, with a true scope and spirit being indicated by the following claims.

1. A method for treating a psychiatric disorder in a subject in need thereof, the method comprising administering to the subject an effective amount of a dosage form comprising:
(a) a therapeutically effective amount of a monoamine oxidase inhibitor (MAOI) selected from phenelzine and tranylcypromine (TCP);
(b) a therapeutically effective amount of a beta blocker selected from pindolol and carvedilol, or a pharmaceutically acceptable salt or free base thereof; and
(c) a pharmaceutically acceptable carrier,
wherein the subject has not previously been diagnosed as having migraines.

2. The method of claim 1, wherein the dosage form consists of the MAOI and the beta blocker.

3. The method of claim 1, wherein the dosage form does not contain an alpha blocker.

4. The method of claim 1, wherein the dosage form does not contain a 5HT1A agonist.

5. (canceled)

6. The method of claim 1, wherein the MAOI is phenelzine.

7. The method of claim 1, wherein the MAOI is TCP.

8. The method of claim 1, wherein the beta blocker is pindolol.

9. The method of claim 1, wherein the beta blocker is carvedilol.

10. The method of claim 1, wherein the subject is not on a tyramine restricted diet subsequent to the administering step.

11. A dosage form comprising:
(a) a therapeutically effective amount of a monoamine oxidase inhibitor (MAOI); and
(b) a therapeutically effective amount of a beta blocker selected from propranolol, metoprolol, carvedilol, timolol, carteolol, atenolol, nebivolol, sotalol, bucindolol, nadolol, celiprolol, nebivolol, betaxolol, esmolol, bisoprolol, oxprenolol, penbutolol, labetalol, acebutolol, and pindolol, or a pharmaceutically acceptable salt or free base thereof, and
a pharmaceutically acceptable carrier.

12. The dosage form of claim 11, wherein the dosage form consists essentially of the MAOI and the beta blocker.

13-17. (canceled)

18. The dosage form of claim 11, wherein the MAOI is formulated as a modified-release dosage form.

19. The dosage form of claim 18, wherein the modified-release dosage form releases the MAOI for a dissolution period lasting from about 6 hours to about 16 hours.

20-27. (canceled)

28. The dosage form of claim 11, wherein the MAOI is present in an amount of from about 30 mg to about 120 mg.

29. The dosage form of claim 11, wherein the MAOI is selected from isocarboxazid, phenelzine, selegiline, tranylcypromine (TCP), and moclobemide.

30-32. (canceled)

33. The dosage form of claim 11, wherein the beta blocker is present in an amount of from about 2.5 mg to about 30 mg.

34-39. (canceled)

40. The dosage form of claim 11, wherein the ratio of the MAOI to the beta blocker is from about 2:1 to about 24:1.

41-50. (canceled)

51. A method for treating a psychiatric disorder in a subject in need thereof, the method comprising administering to the subject an effective amount of the dosage form of claim 11.

52-72. (canceled)

73. A method for treating a psychiatric disorder in a subject in need thereof, the method comprising administering to the subject:
(a) a therapeutically effective amount of a MAOI; and
(b) a therapeutically effective amount of a beta blocker selected from propranolol, metoprolol, carvedilol, timolol, carteolol, atenolol, nebivolol, sotalol, bucindolol, nadolol, celiprolol, nebivolol, betaxolol, esmolol, bisoprolol, oxprenolol, penbutolol, labetalol, acebutolol, and pindolol, or a pharmaceutically acceptable salt or free base thereof,
wherein the subject has not previously been diagnosed as having migraines, and
wherein the subject is not currently experiencing a migraine.

74-97. (canceled)
